(19) United States
(12) Patent Application Publication
(10) Pub. No.: US 2018/0371088 A1
(43) Pub. Date: Dec. 27, 2018

(54) TARGET CELL-DEPENDENT T CELL ENGAGING AND ACTIVATION ASYMMETRIC HETERODIMERIC Fc-ScFv FUSION ANTIBODY FORMAT AND USES THEREOF IN CANCER THERAPY

(71) Applicant: Development Center for Biotechnology, New Taipei City (TW)

(72) Inventors: Chia-Cheng WU, New Taipei City (TW); Tzu-Yin LIN, New Taipei City (TW); Chao-Yang HUANG, New Taipei City (TW); Yu-Jung CHEN, New Taipei City (TW); Jei-Hwa YU, New Taipei City (TW); Chen-Li CHIEN, New Taipei City (TW)

(73) Assignee: Development Center for Biotechnology, New Taipei City (TW)

(21) Appl. No.: 16/016,414

(22) Filed: Jun. 22, 2018

Related U.S. Application Data
(60) Provisional application No. 62/523,279, filed on Jun. 22, 2017.

(51) Int. Cl. C07K 16/28; C07K 16/30; A61P 35/00

(52) U.S. Cl. CPC C07K 16/2809; C07K 16/3076; C07K 16/3015; C07K 2317/622; C07K 2317/526; C07K 2317/71; C07K 2317/31; A61P 35/00

(57) ABSTRACT

An asymmetric heterodimeric antibody includes a knob structure formed in a CH3 domain of a first heavy chain; a hole structure formed in a CH3 domain of a second heavy chain, wherein the hole structure is configured to accommodate the knob structure so that a heterodimeric antibody is formed; and a T-cell targeting domain fused to the CH3 domain of the first heavy chain or the second heavy chain, wherein the T-cell targeting domain binds specifically to an antigen on the T-cell. The T-cell targeting domain is a ScFv or Fab derived from an anti-CD3 antibody. The asymmetric heterodimeric antibody may have L234A and L235A mutations or L235A and G237A such that its effector binding is compromised.

### BACKGROUND OF INVENTION

### Field of the Invention

The present invention relates to antibody engineering, particularly to asymmetric heterodimeric antibodies that are multi-specific.

### Background Art

Multi-specific antibodies (e.g., bispecific antibodies) are promising therapeutics for diseases. Asymmetric bispecific antibodies are designed to recognize two different target epitopes. These antibodies can achieve novel functions that are not achievable with conventional antibodies. One approach to asymmetric bispecific antibodies is to design knob-and-hole in the CH3 domains of the heavy chains. The complementarity of knob-and-hole structure favors the formation of heterodimer antibodies. (A. M. Merchant et al., “An efficient route to human bispecific IgG,” Nat. Biotechnol., 1998, 16:677-81; doi: 10.1038/nbt0798-677).

Asymmetric bispecific antibodies have shown potential applications in therapy. However, there is still a need for better asymmetric antibodies that are multi-specific.

### SUMMARY OF INVENTION

The present invention relates to a platform for the generation of asymmetric antibodies, which may have multi-specificities, and their uses in therapy.

In accordance with embodiments of the invention, an asymmetric antibody may have heavy chains comprising a knob arm and a hole arm. These antibodies have heterodimeric Fc-ScFv (AHFS) or Fab (AHFF) fusion bispecific or trispecific antibody format, wherein the ScFv or Fab are derived from a T-cell targeting antibody, such as an anti-CD3 antibody. The ScFv or Fab may be fused either to the knob arm or to the hole arm.

In accordance with embodiments of the invention, to diminish ADCC and CDC effector functions, the amino acid residues in the CH2 domains of both knob arm and the hole arm may contain mutations. For example, residues at positions 234 and 235 may be changed from leucine to alanine, or residues at positions 235 and 237 may be changed from leucine and glycine to alanine. Similarly, other approaches to reducing/eliminating the effector functions known in the art may also be used.

In accordance with embodiments of the invention, to enhance Fc heterodimerization, the two halves of an antibody may be engineered to have complementary structures such that they will bind preferably to each other to form an asymmetric dimmer. Such approaches known in the art include the “knob-into-hole” approach, which involves constructing a “knob” in one of the heavy chain CH3 domain and a “hole” in the other heavy chain CH3 domain. For example, the amino acid residues of the knob arm's CH3 domain at position 354 and 366 may be changed from serine and threonine to cysteine and tryptophan, and the amino acid residues of the hole arm's CH3 domain at position 349, 366, 368 and 407 may be changed from tyrosine, threonine, leucine and tyrosine to cysteine, serine, alanine and valine, respectively. T cell engaging and activation by an antibody of the invention is dependent on the presence of antigens expressing on the surface of target cells.

One aspect of the invention relates to asymmetric heterodimeric antibodies. An asymmetric heterodimeric antibody in accordance with one embodiment of the invention includes a knob structure formed in a CH3 domain of a first heavy chain; a hole structure formed in a CH3 domain of a second heavy chain, wherein the hole structure is configured to accommodate the knob structure so that a heterodimeric antibody is formed; and a T-cell targeting domain fused to the CH3 domain of the first heavy chain or the second heavy chain, wherein the T-cell targeting domain binds specifically to an antigen on the T-cell.

In accordance with some embodiments of the invention, the T-cell targeting domain may be a ScFv or Fab. In accordance with some embodiments of the invention, the ScFv or the Fab may be derived from an anti-CD3 antibody.

In accordance with some embodiments of the invention, the asymmetric heterodimeric antibody may have its effector binding site mutated such that it has a diminished binding to an effector cell. The asymmetric heterodimeric antibody with diminished effector binding may have L234A and L235A mutations or L235A and G237A mutations in the CH2 domains.

Another aspect of the invention relates to methods for treating cancers. A method in accordance with one embodiment of the invention comprises administering to a subject in need thereof any one of the above-described asymmetric heterodimeric antibodies.

Other aspect of the invention will become apparent with the following description.

### BRIEF DESCRIPTION OF THE DRAWINGS

FIG. 1A shows a schematic illustrating a generic format of an asymmetric heterodimeric antibody of the invention. FIG. 1B shows a schematic illustrating embodiment of the invention having Fab as binders. FIG. 1C shows a schematic illustrating embodiment of the invention having ScFv as binders. FIG. 1D shows a schematic illustrating embodiment of the invention having growth factors or cytokines as binders. FIG. 1E shows a schematic illustrating embodiment of the invention having cancer targeting peptides as binders.

FIG. 2A shows various expression vector constructs for the production of “knob” arms of different formats of asymmetric dimeric multi-specific antibodies in accordance with embodiments of the invention. FIG. 2B shows various expression vector constructs for the production of “knob” arms of different formats of asymmetric dimeric multi-specific antibodies, which contain mutations in the effector binding sites, in accordance with embodiments of the invention. FIG. 2C shows various expression vector constructs for the production of “hole” arms of different formats of asymmetric dimeric multi-specific antibodies in accordance with embodiments of the invention. FIG. 2D shows various expression vector constructs for the production of “hole” arms of different formats of asymmetric dimeric multi-specific antibodies, which contain mutations in the effector binding sites, in accordance with embodiments of the invention. FIG. 2E shows various expression vector constructs for the production of heavy chain without the T-cell targeting domains of different formats of asymmetric dimeric multi-specific antibodies, which contain mutations in the effector binding sites, in accordance with embodiments of the invention.

FIG. 3 shows that AHFS of the invention, with or without mutations at the effector binding site, can bind specifically to Jurkat T cells when a T-cell targeting domain is present.

FIG. 4 shows that AHFS of the invention, with or without mutations at the effector binding site, can bind specifically to breast cancer cells HC1428 when a T-cell targeting domain is present.

FIG. 5 shows that AHFS EGF×anti-CD3 and breast cancer targeting peptide (CTP)×anti-CD3 bispecific proteins but not AHFS AMG386×anti-CD3 bispecific protein bind to breast cancer BT474 target cells.

FIG. 6 shows that AHFS Anti-TAA×anti-CD3 bispecific antibody effectively kills TAA expressing breast cancer cell line HCC1428 in the presence of human PBMC and in the absence of ADCC function.

FIG. 7 shows that AHFS Anti-TAA×anti-CD3 bispecific antibody effectively kills TAA expressing breast cancer cell line HCC1428 in the presence of T cells.

FIG. 8 shows that AHFS N-LFv×anti-CD3 bispecific and trispecific antibodies effectively kills TAA and HER2 expressing breast cancer cell line HCC1428 in the presence of T cells.

FIG. 9 shows that AHFS N-LFv×anti-CD3 bispecific and trispecific antibodies effectively kills HER2 expressing breast cancer cell line BT474 in the presence of T cells.

FIG. 10 shows that AHFS N-ScFv×anti-CD3 bispecific antibodies effectively kills HER2 expressing breast cancer cell line HCC1428 in the presence of T cells.

FIG. 11 shows that AHFS EGF×anti-CD3 bispecific proteins effectively kills HER2 expressing breast cancer cell line BT474 in the presence of T cells.

FIG. 12 shows that AHFS breast cancer CTP×anti-CD3 bispecific proteins effectively kills breast cancer cell line BT474 in the presence of T cells.

FIG. 13 shows that IL-2 production by NK cell and Non-specific T cell activation induced by Fc-anti-CD3 ScFv fusion domain were completely diminished by Fc engineering of L234A and L235A or L235A and G237A.

FIG. 14 shows that TNF-α production by NK cell and Non-specific T cell activation induced by Fc-anti-CD3 ScFv fusion domain were completely diminished by Fc engineering of L234A and L235A or L235A and G237A.

FIG. 15 shows that NK cell activation and IFN-γ production were completely diminished by Fc engineering of L234A and L235A or L235A and G237A.

FIG. 16 shows that Granzyme B production by NK cell and Non-specific T cell activation induced by Fc-anti-CD3 ScFv fusion domain were completely diminished by Fc engineering of L234A and L235A or L235A and G237A.

FIG. 17 shows that Perforin production by NK cell and Non-specific T cell activation induced by Fc-anti-CD3 ScFv fusion domain were completely diminished by Fc engineering of L234A and L235A or L235A and G237A.

FIG. 18 shows that AHFS anti-TAA×anti-CD3 BsAb effectively activates T cell and induces IL-2 production in a tumor target cell-dependent manner.

FIG. 19 shows that AHFS anti-TAA×anti-CD3 BsAb effectively activates T cell and induces TNF-α production in a tumor target cell-dependent manner.

FIG. 20 shows that enhancement of IFN-γ production by Fc-anti-CD3 ScFv fusion and engineering of L234A and L235A or L235A and G237A.

FIG. 21 shows that enhancement of Granzyme B production by Fc-anti-CD3 ScFv fusion and engineering of L234A and L235A or L235A and G237A.

FIG. 22 shows that AHFS anti-TAA×anti-CD3 BsAb effectively activates T cell and induces Perforin production in a tumor target cell-dependent manner.

### DETAILED DESCRIPTION

Embodiments of the invention relate to methods for the production of multi-specific asymmetric antibodies and uses thereof. In accordance with embodiments of the invention, an asymmetric antibody contains two heavy chains that are not identical. One of the heavy chain functions as a knob arm, and the other heavy chain functions as a hole arm that can accommodate the knob. The knob and hole structures are engineered (e.g., by site-directed mutagenesis) in the third constant domain of the heavy chains, CH3. The complementarity of the knob and hole facilitates the formation of asymmetric antibodies.

In accordance with embodiments of the invention, to enhance Fc heterodimerization, the amino acid residues of the knob arm CH3 domain at positions 354 and 366 are changed from serine and threonine to cysteine and tryptophan, respectively, and the amino acid residues of the hole arm CH3 domain at positions 349, 366, 368 and 407 are changed from tyrosine, threonine, leucine and tyrosine to cysteine, serine, alanine and valine, respectively. While specific examples of knob-into-hole asymmetric antibodies are illustrated in this description, other similar mutations known in the art may also be used without departing from the scope of the invention. (A. M. Merchant et al., “An efficient route to human bispecific IgG,” Nat. Biotechnol., 1998, 16:677-81; doi: 10.1038/nbt0798-677; and A. Tustian et al., “Development of purification processes for fully human bispecific antibodies based upon modification of protein A binding avidity,” MAbs, 2016 May-June; 8(4): 828-838; doi: 10.1080/19420862.2016.1160192.)

Some embodiments of the invention are bispecific antibodies that include asymmetric antibodies (heterodimeric antibodies) containing two different antigen-binding domains. Some embodiments of the invention are multi-specific antibodies that contain more than two different antigen-binding domains.

For example, some embodiments of the invention may be tri-specific antibodies in the form of heterodimeric Fc-ScFv (AHFS) or heterodimeric Fc-Fab (AHFF) fusion antibody formats, wherein the ScFv or Fab may be derived from any antibody selected for T-cell targeting, for example anti-CD3 antibodies. In accordance with embodiments of the invention, the ScFv or the Fab fragments may be fused with either the knob arm or the hole arm of the antibodies to produce tri-specific antibodies. In accordance with other embodiments of the invention, different ScFv or the Fab fragments may be fused with both the knob arm and the hole arm of the antibodies to produce tetra-specific antibodies.

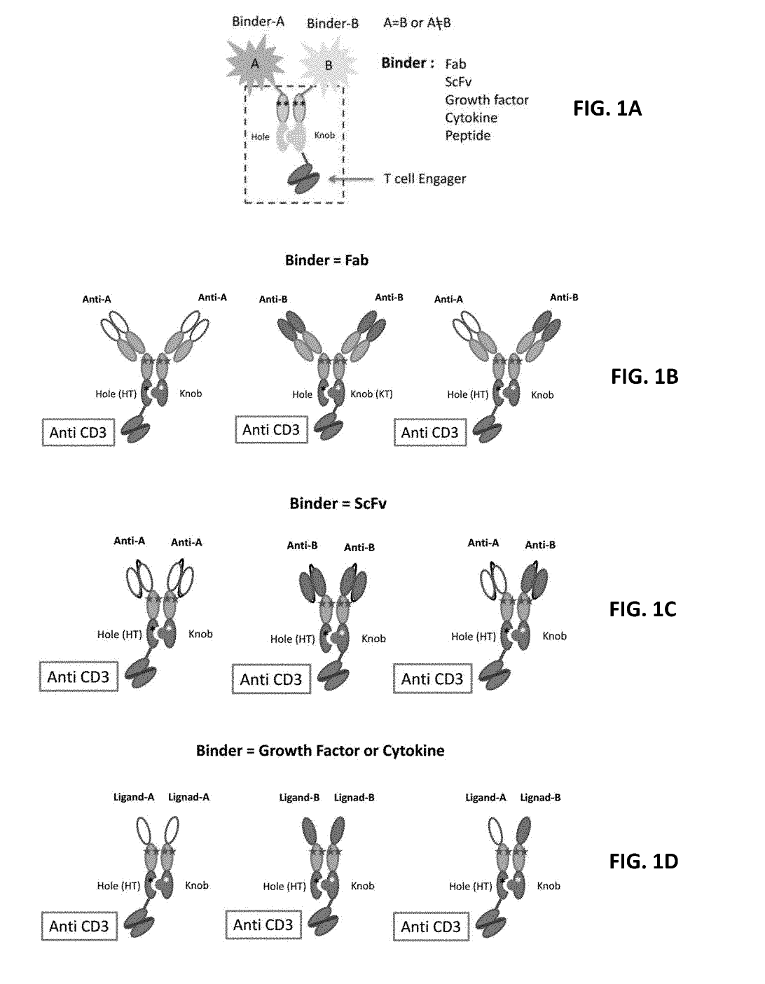

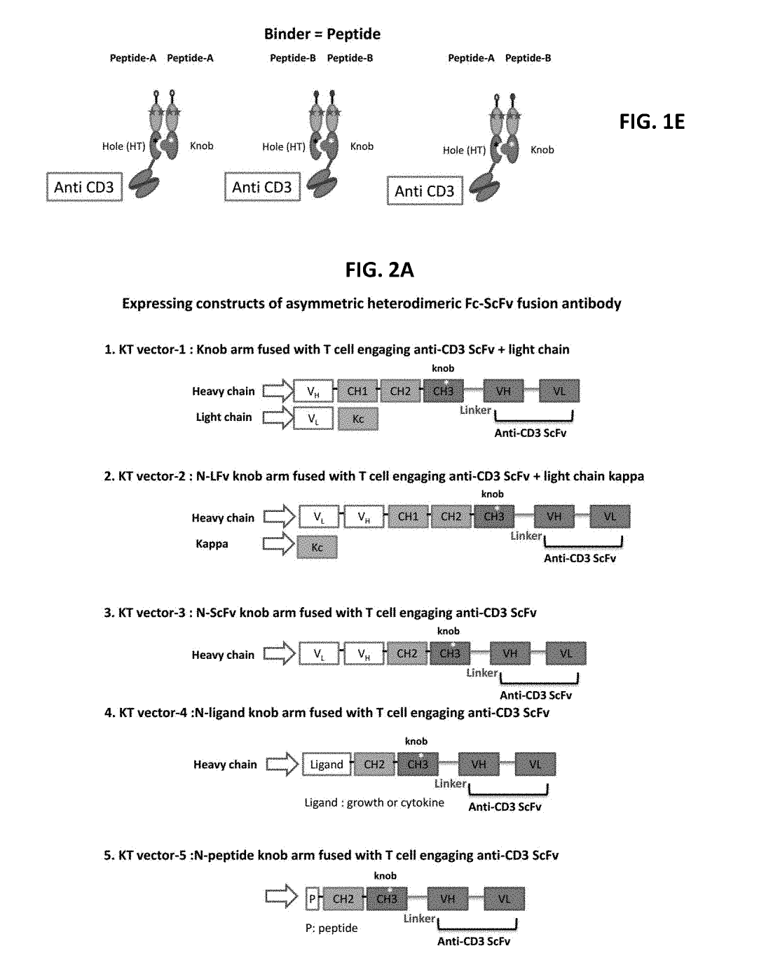

FIG. 1A shows a schematic of a generic form of an asymmetric antibody of the invention. As shown, the antibody has binders A and B located at where the variable domains of a typical antibody will be. The A and B binders may be identical or different. They may comprise an Fab, an ScFv, a growth factor, a cytokine, or a peptide. In addition, a T-cell engager (i.e., a T-cell targeting domain) is fused to one of the CH3 domain. The T-cell engager, for example, may be an ScFv or Fab derived from an anti-CD3 antibody.

The Anti-A and Anti-B shown in FIG. 1A may be selected for any desired target. For example, for cancer therapy, such antigens may be selected for a tumor-associated antigen (TAA), such as Her2, alpha-enolase, etc.

FIG. 1B shows schematics illustrating three different possibilities when the T-cell engager is derived from an anti-CD3 antibody, and the binders A and B are Fab fragments, which may be identical or different (i.e., anti-A+anti-A; anti-B+anti-B; or anti-A+anti-B).

FIG. 1C shows schematics illustrating three different possibilities when the T-cell engager is derived from an anti-CD3 antibody, and the binders A and B are ScFv fragments, which may be identical or different.

FIG. 1D shows schematics illustrating three different possibilities when the T-cell engager is derived from an anti-CD3 antibody, and the binders A and B are growth factors or cytokines, which may be identical or different.

FIG. 1E shows schematics illustrating three different possibilities when the T-cell engager is derived from an anti-CD3 antibody, and the binders A and B are peptides that can target specific binding sites (e.g., receptors), which may be identical or different.

In these examples, an anti-CD3 ScFv is illustrated as the T-cell targeting domain (T-cell engager). One skilled in the art would appreciate that these examples are for illustration only and other T-cell targeting binders may also be used without departing from the scope of the invention.

While antibody effector functions, such as antibody-dependent cellular cytotoxicity (ADCC) and complement-dependent cytotoxicity (CDC), are desirable in immune therapies, these effector functions sometimes are not desirable. Thus, some therapeutic antibodies may prefer to have reduced or silenced effector functions.

For example, multi-specific antibodies of the invention may contain a binding site directed to a target on immune cells (see e.g., anti-CD3 in FIG. 1), while another binding site may be targeting a tumor associated antigen (TAA). If the effector function is intact, the NK cells (via FcR on NK cells) can bind to the effector function site (located in in the hinge and CH2 domain) of Fc portion of the multi-specific antibody, while anti-CD3 binds CD3 on T-cells. When this occurs, the NK cells may mediate cytotoxicity towards the T cells. This would be counterproductive.

Several approaches have been disclosed in the prior art for reducing the effector functions, including glycan modifications, use of IgG2 or IgG4 subtype that do not interact well with the receptors on the effector cells, or mutations in the effector interaction sites (i.e., the lower hinges or CH2 domains) of the bispecific antibodies.

In accordance with embodiments of the invention, some antibodies may be modified to reduce or diminish ADCC and CDC effector functions. In one example, amino acid residues at positions 234 and 235 in the CH2 domains of the knob arms and/or the hole arms are changed from leucine to alanine. In another example, amino acid residues at positions 235 and 237 in the CH2 domains of the knob arms and/or the hole arms are changed from leucine and glycine to alanine.

With diminished ADCC and CDC effector functions, such antibodies will not trigger ADCC or CDC response on its own. Instead, T-cells engagement and activation is dependent on binding of antibodies of the invention to the antigens expressed on the surfaces of target cells, thereby increasing the efficiency of targeted therapy.

Antibodies of the invention may be obtained with various expression constructs. The modifications of the expression vectors and the expressions of these constructs involve routine techniques known in the art. One skilled in the art would be able to construct these expression vectors and obtain expressed proteins without undue experimentation.

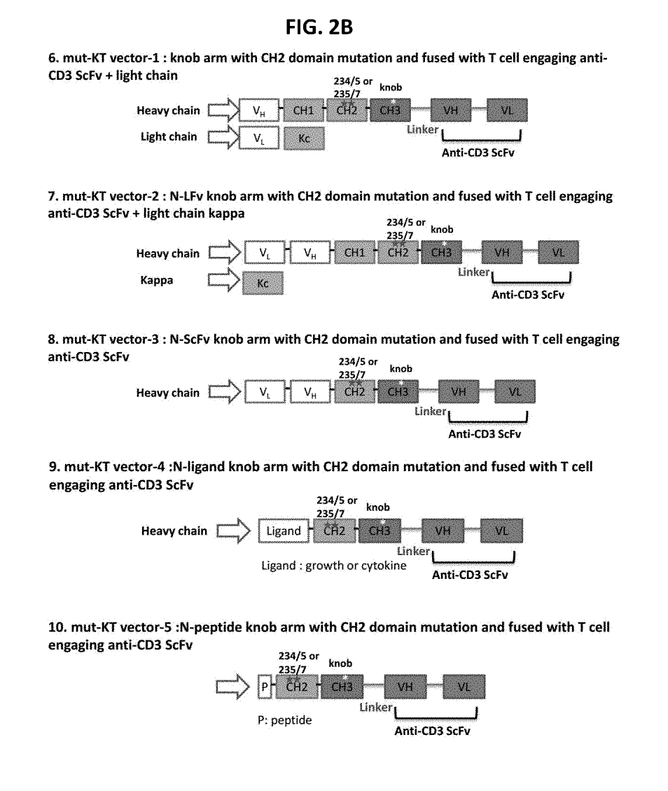

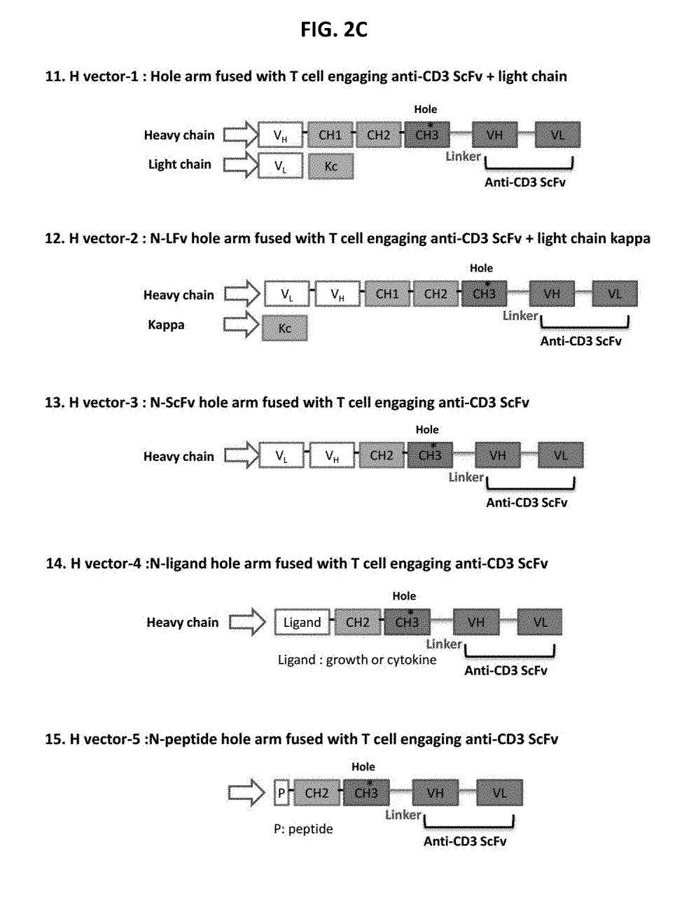

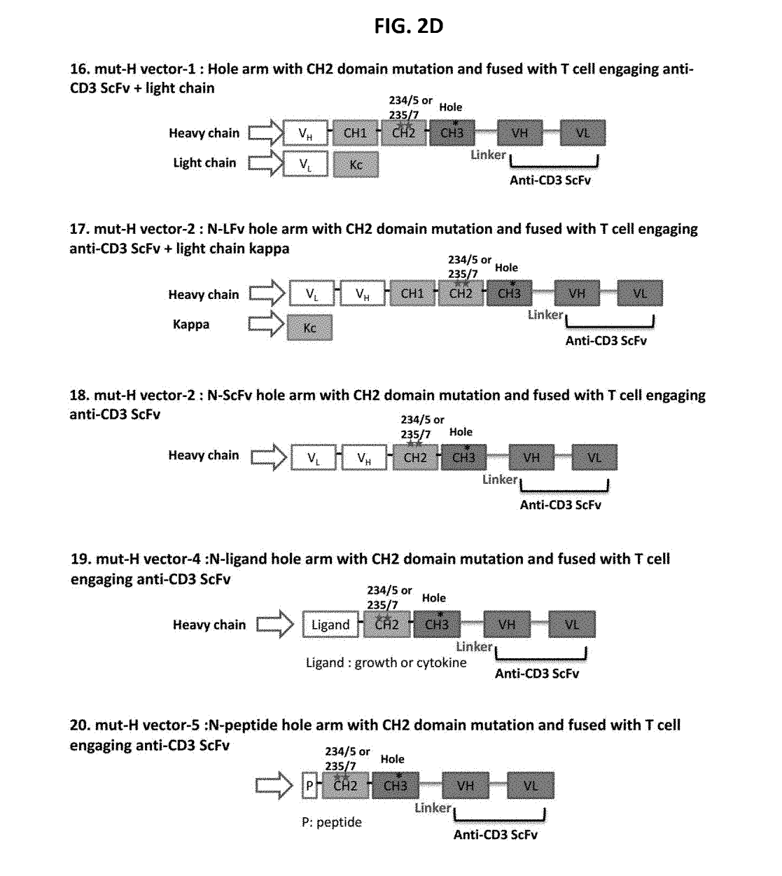

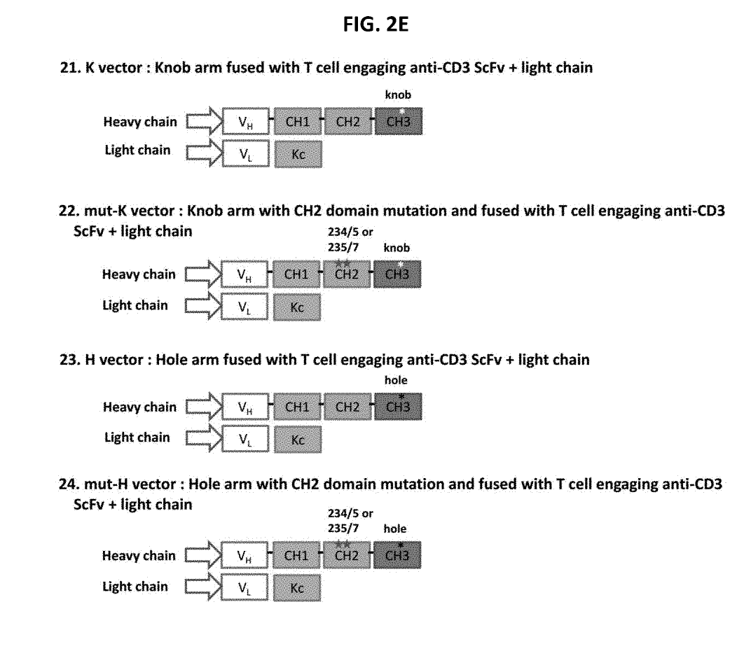

FIG. 2A shows various expression constructs for asymmetric heterodimeric Fc-ScFv fusion antibodies (KT vectors, i.e., Knob arm containing the Tethered binding fragment, anti-CD3 ScFv). In these examples, the heavy-chain expression vectors contain modifications in the CH3 domains to form the “knob” structures. In addition, an anti-CD3 ScFv is fused to the C-terminus of the heavy chain.

As shown in FIG. 2A, KT vector-1 contains a heavy-chain variable domain (as in a regular antibody), which can associate with a light chain to form a binding domain (i.e., binder A or binder B in FIG. 1A). KT vector-2 contains a light-chain variable domain (LFv) fused with a heavy-chain variable domain to form a binding domain. In this construct, the heavy-chain retains the first constant domain. Therefore, a light chain constant domain (e.g., a kappa chain) may associate with this heavy-chain fusion protein. KT vector-3 contains a light-chain variable domain (LFv) fused with a heavy-chain variable domain, in a format of an ScFv, to form a binding domain (i.e., binder A or binder B in FIG. 1A). In this construct, the heavy chain lacks the first constant domain. Therefore, a light chain constant domain will not associate with this fusion protein. KT vector-4 and KT vector-5 contain a ligand (e.g., a growth factor or cytokine) or a peptide, respectively fused with heavy-chain constant domains. The ligand or peptide is selected for specific binding with a target (e.g., a receptor on tumor cells), while the T-cell targeting domain (e.g., the anti-CD3 ScFv) can bind the T cells.

FIG. 2B shows various expression constructs for asymmetric heterodimeric Fc-ScFv fusion antibodies that also include mutations at the effector binding sites. In these examples (mut-KT vector, i.e., Knob arm containing the Tethered binding fragment, anti-CD3 ScFv, and the CH2 domain contains the mutations to reduce or eliminate the effector functions), the heavy-chain expression vector contains modifications in the CH3 domain to form the “knob” structure and mutations in the CH2 domain to compromise the effector functions. In addition, an anti-CD3 scFv is fused to the C-terminus of the heavy chain. As compared with the constructs shown in FIG. 2A, these mutant constructs (mutations at the effector biding sites) will have no or diminished effector functions. As a result, there will be minimal or no non-specific T-cell activation. The T-cells bound by the bispecific or multispecific antibodies of the invention will be activated only after the binding domains bind to the target cells (e.g., tumor cells).

The above examples show the knob arms of the antibody heavy chains. The corresponding “hole” arms can be similarly constructed. FIG. 2C and FIG. 2D show expression constructs for the “hole” arms corresponding to those in FIG. 2A and FIG. 2B, respectively. In the examples shown in FIG. 2C (HT vectors, i.e., Hole arm containing the Tethered binding fragment, anti-CD3 ScFv), the heavy-chain expression vectors contain modifications in the CH3 domains to form the “hole” structures. In addition, an anti-CD3 ScFv is fused to the C-terminus of the heavy chain.

In the examples shown in FIG. 2D (mut-HT vector, i.e., Hole arm containing the Tethered binding fragment, anti-CD3 ScFv, and the CH2 domain contains the mutations to reduce or eliminate the effector functions), the heavy-chain expression vector contains modifications in the CH3 domain to form the “hole” structure and mutations in the CH2 domain to compromise the effector functions. In addition, an anti-CD3 ScFv is fused to the C-terminus of the heavy chain.

In the above examples, the heavy chain CH3 is fused with a T-cell targeting domain (e.g., Anti-CD3 ScFv). To form an asymmetric antibody, the proteins from the above constructs may be paired with proteins from constructs without the fused anti-CD3 ScFv. FIG. 2E shows exemplary constructs for producing proteins, with or without mutations in CH2 domains, with the anti-CD3 ScFv.

These expression vectors may be transfected into any suitable cells for antibody expression, such as CHO cells, 293 cells, etc. Methods for the expressions and purifications of the antibodies are known in the art.

A general outline for the production of an asymmetric antibody of the invention may be as follows: (1) A heavy chain N-terminal binder region and a light chain N-terminal binder region of these vector are engineered from the V_{H} and V_{L} of any tumor associated antigen (TAA) specific antibodies or receptor ligands, such as growth factors, cytokines or cancer targeting peptides (CTP). (2) asymmetric heterodimeric Fc-ScFv bispecific or trispecific antibody may be generated by co-transfection of either heavy chain native or modified(mut) KT and H (KT+H) plasmid DNAs or K and HT (K+HT) plasmid DNAs into production cell host, such as 293-FS or CHO cells. (3) to generate bispecific antibody, V_{H} and V_{L} of heavy chain native or modified (mut) KT and H (KT+H) plasmid DNA or K and HT (K+HT) plasmid DNA are engineered from the same antibody. (4) to generation trispecific antibody, V_{H} and V_{L} of heavy chain native or modified (mut) KT and H (KT+H) plasmid DNA or K and HT (K+HT) plasmid DNA are engineered from two different antibodies. (5) amino acid residue modifications of heavy chain CH2 domain are L234A and L235A or L235A, G237A. (6) amino acid residue modification of heavy chain knob arm CH3 domain are S354C and T366W. (7) amino acid residue modification of heavy chain hole arm CH3 domain are Y349C, T366S, L368A, Y407V. (8) ScFv of KT or HT vector is engineered from anti-CD3 antibodies. (9) ScFv of KT or HT vector can be replaced by Fab of anti-CD3 antibodies.

Method of generating an asymmetric heterodimeric Fc-ScFv (AHFS) fusion antibody may be as follows: (1) knob arm and hole arm are generated by subcloning of PCR amplified synthetic knob arm gene, S354C and T366W, and hole arm gene, Y349C, T366S, L368A, and Y407V, with MfeI and BamHI digestion, and into targeted antibody expressing pTACE8 vector. (2) knob arm or hole arm fused with anti-CD3 ScFv are generated by assembly PCR of synthetic knob arm-linker or hole arm-liker gene fragment with linker-anti-CD3 ScFv gene fragment and the assembled DNA following MfeI and BamHI digestion are subcloned into target antibody expressing vector, and subcloning of the whole heavy chain fragment into different target antibody expressing vector was digested with AvrII and BstZ17I. (3) mutations of CH2 domain are generated by assembly PCR of synthetic gene fragment with L234A and L235A mutation or L235A and G237A mutation, and the assembled DNAs, following NheI and MfeI digestion, are subcloned into target antibody expressing vector, and subcloning of the whole heavy chain fragment into different target antibody expressing vector is digested with AvrII and BstZ17I

Embodiments of the invention will be illustrated with the following specific examples. One skilled in the art would appreciate that these examples are for illustration only and that other modifications and variations are possible without departing from the scope of the invention. Various molecular biology techniques, vectors, expression systems, protein purification, antibody-antigen binding assays, etc. are well known in the art and will not be repeated in details.

### EXAMPLES

### Example 1

### Preparation of Anti-TAA Antibody

In accordance with embodiments of the invention, a general method for the generation of monoclonal antibodies includes obtaining a hybridoma producing a monoclonal antibody against a selected TAA. Alternatively, the multi-specific asymmetric antibodies of the invention may be obtained starting from a known monoclonal antibody, for example the anti-Her2 antibody trastuzumab.

Methods for the production of monoclonal antibodies are known in the art and will not be elaborated here. Briefly, mice are challenged with the antigen (TAA) with an appropriate adjuvant. Then, the spleen cells of the immunized mice were harvested and fused with myeloma. Positive clones may be identified for their abilities to bind TAA, using any known methods, such as ELISA.

In accordance with embodiments of the invention, sequences of the antibodies are determined and used as the basis for mutations to generate knob and hole structures, as well as mutations to reduce or silence the effector functions. Briefly, the total RNA of the hybridoma was isolated, for example using the TRIzol® reagent. Then, cDNA was synthesized from the total RNA, for example using a first strand cDNA synthesis kit (Superscript III) and an oligo(d_{T20}) primer or an Ig-3′ constant region primer. Heavy and light chain sequences of the immunoglobulin genes were then cloned from the cDNA. The cloning may use PCR, using appropriate primers, e.g., Ig-5′ primer set (Novagen). The PCR products may be cloned directly into a suitable vector (e.g., a pJET1.2 vector) using CloneJet™ PCR Cloning Kit (Fermentas). The pJET1.2 vector contains lethal insertions and will survive the selection conditions only when the desired gene is cloned into this lethal region. This facilitates the selection of recombinant colonies. Finally, the recombinant colonies were screened for the desired clones, the DNAs of those clones were isolated and sequenced. The immunoglobulin (IG) nucleotide sequences may be analyzed at the international ImMunoGeneTics information system (IGMT) website. The CDR sequences may be identified using Kabat method.

### Example 2

### Mutagenesis to Generate Knob-and-Hole Structures and to Silence the Effector Functions

The anti-TAA monoclonal antibody sequences are used as basis for site-directed mutagenesis using techniques known in the art, such as using PCR. The desired mutant clones can be confirmed with sequencing analysis.

As a particular example using an anti-TAA antibody sequences, the nucleotide and amino acid sequences of an asymmetric heterodimer ScFv (AHFS) IgG Hole arm with L234A, L235A , Y349C, T366S, L368A, and Y407V mutations are as follows:

(SEQ ID NO: 1)
gctagcaccaagggcccttccgtgttccctctggccccttccagcaagtc
tacctctggcggcaccgctgctctgggctgcctcgtgaaggactacttcc
ctgagcctgtgacagtgtcctggaactaggcgccctgacctccggcgtgc
acaccttccctgccgtgctgcagtcctccggcctgtactactgtcctccg
tcgtgacagtgccttcctccagcctgggcacccagacctacatctgcaac
gtgaaccacaagccttccaacaccaaggtggacaagaaggtggagcctaa
gtcctgcgacaagacccacacctgtcctccatgccctgcccctgaggctg
ctggcggaccctccgtgttcctgttccctccaaagcctaaggacaccctg
atgatctcccggacccctgaagtgacctgcgtggtggtggacgtgtccca
cgaggaccctgaagtgaagttcaattggtacgtggacggcgtggaagtgc
acaacgccaagaccaagcccagagaggaacagtacaactccacctaccgg
gtggtgtccgtgctgaccgtgctgcaccaggattggctgaacggcaaaga
gtacaagtgcaaggtgtccaacaaggccctgcctgcccccatcgaaaaga
ccatctccaaggccaagggccagccccgggaacctcaagtgtgcaccctg
ccccctagccgggaagagatgaccaagaaccaggtgtccctgtcctgcgc
cgtgaagggcttctacccctccgacattgccgtggaatgggagtccaacg
gccagcctgagaacaactacaagaccaccccccctgtgctggactccgac
ggctcattcttcctggtgtccaagctgacagtggacaagtcccggtggca
gcagggcaacgtgttctcctgaccgtgatgcacgaggccctgcacaacca
ctacacccagaagtccctgagcctgtcccaggc.
(SEQ ID NO: 15)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVCTLPPSREEMTKNQVSLSC
AVKGFYPSDIAVEWESNGQPENNYKTTPPVLDSDGSFFLVSKLTVDKSRW
QQGNVFSCSVMHEALHNHYTQKSLSLSPG.

The nucleotide and amino acid sequences of an AHFS IgG Knob arm with L234A, L235A, S354C and T366W mutations are as follows:

(SEQ ID NO: 2)
gctagcaccaagggcccttccgtgttccctctggccccttccagcaagtc
tacctctggcggcaccgctgctctgggctgcctcgtgaaggactacttcc
ctgagcctgtgacagtgtcctggaactaggcgccctgacctccggcgtgc
acaccttccctgccgtgctgcagtcctccggcctgtactactgtcctccg
tcgtgacagtgccttcctccagcctgggcacccagacctacatctgcaac
gtgaaccacaagccttccaacaccaaggtggacaagaaggtggagcctaa
gtcctgcgacaagacccacacctgtcctccatgccctgcccctgaggctg
ctggcggaccctccgtgttcctgttccctccaaagcctaaggacaccctg
atgatctcccggacccctgaagtgacctgcgtggtggtggacgtgtccca
cgaggaccctgaagtgaagttcaattggtacgtggacggcgtggaagtgc
acaacgccaagaccaagcccagagaggaacagtacaactccacctaccgg
gtggtgtccgtgctgaccgtgctgcaccaggattggctgaacggcaaaga
gtacaagtgcaaggtgtccaacaaggccctgcctgcccccatcgaaaaga
ccatctccaaggccaagggccagccccgggaaccccaggtgtacacactg
cccccttgccgggaagagatgaccaagaaccaggtgtccctgtggtgcct
cgtgaagggcttctacccctccgacattgccgtggaatgggagtccaacg
gccagcctgagaacaactacaagaccaccccccctgtgctggactccgac
ggctcattcttcctgtactccaagctgacagtggacaagtcccggtggca
gcagggcaacgtgttctcctgaccgtgatgcacgaggccctgcacaacca
ctacacccagaagtccctgtccctgagccctggc.
(SEQ ID NO: 16)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPCREEMTKNQVSLWC
LVKGFYPSDIAVEWESNGQPENNYKTTPPVLDSDGSFFLYSKLTVDKSRW
QQGNVFSCSVMHEALHNHYTQKSLSLSPG.

Similarly, the nucleotide and amino acid sequences of an AHFS IgG Hole arm with L235A, G237A, Y349C, T366S, L368A, and Y407V mutations are as follows:

(SEQ ID NO: 3)
gctagcaccaagggcccttccgtgttccctctggccccttccagcaagtc
tacctctggcggcaccgctgctctgggctgcctcgtgaaggactacttcc
ctgagcctgtgacagtgtcctggaactaggcgccctgacctccggcgtgc
acaccttccctgccgtgctgcagtcctccggcctgtactactgtcctccg
tcgtgacagtgccttcctccagcctgggcacccagacctacatctgcaac
gtgaaccacaagccttccaacaccaaggtggacaagaaggtggagcctaa
gtcctgcgacaagacccacacctgtcctccatgccctgcccctgagctgg
ctggcgctccctccgtgttcctgttccctccaaagcctaaggacaccctg
atgatctcccggacccctgaagtgacctgcgtggtggtggacgtgtccca
cgaggaccctgaagtgaagttcaattggtacgtggacggcgtggaagtgc
acaacgccaagaccaagcccagagaggaacagtacaactccacctaccgg
gtggtgtccgtgctgaccgtgctgcaccaggattggctgaacggcaaaga
gtacaagtgcaaggtgtccaacaaggccctgcctgcccccatcgaaaaga
ccatctccaaggccaagggccagccccgggaacctcaagtgtgcaccctg
ccccctagccgggaagagatgaccaagaaccaggtgtccctgtcctgcgc
cgtgaagggcttctacccctccgacattgccgtggaatgggagtccaacg
gccagcctgagaacaactacaagaccaccccccctgtgctggactccgac
ggctcattcttcctggtgtccaagctgacagtggacaagtcccggtggca
gcagggcaacgtgttctcctgaccgtgatgcacgaggccctgcacaacca
ctacacccagaagtccctgagcctgtcccctggc.
(SEQ ID NO: 17)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVCTLPPSREEMTKNQVSLSC
AVKGFYPSDIAVEWESNGQPENNYKTTPPVLDSDGSFFLVSKLTVDKSRW
QQGNVFSCSVMHEALHNHYTQKSLSLSPG.

The nucleotide and amino acid sequences of an AHFS IgG Knob arm with L235A, G237A, S354C and T366W mutations are as follows:

(SEQ ID NO: 4)
gctagcaccaagggcccttccgtgttccctctggccccttccagcaagtc
tacctctggcggcaccgctgctctgggctgcctcgtgaaggactacttcc
ctgagcctgtgacagtgtcctggaactaggcgccctgacctccggcgtgc
acaccttccctgccgtgctgcagtcctccggcctgtactactgtcctccg
tcgtgacagtgccttcctccagcctgggcacccagacctacatctgcaac
gtgaaccacaagccttccaacaccaaggtggacaagaaggtggagcctaa
gtcctgcgacaagacccacacctgtcctccatgccctgcccdgagctggc
tggcgctccctccgtgttcctgttccctccaaagcctaaggacaccctga
tgatctcccggacccctgaagtgacctgcgtggtggtggacgtgtcccac
gaggaccctgaagtgaagttcaattggtacgtggacggcgtggaagtgca
caacgccaagaccaagcccagagaggaacagtacaactccacctaccggg
tggtgtccgtgctgaccgtgctgcaccaggattggctgaacggcaaagag
tacaagtgcaaggtgtccaacaaggccctgcctgcccccatcgaaaagac
catctccaaggccaagggccagccccgggaaccccaggtgtacacactgc
ccccttgccgggaagagatgaccaagaaccaggtgtccctgtggtgcctc
gtgaagggcttctacccctccgacattgccgtggaatgggagtccaacgg
ccagcctgagaacaactacaagaccaccccccctgtgctggactccgacg
gctcattcttcctgtactccaagctgacagtggacaagtcccggtggcag
cagggcaacgtgttctcctgctccgtgatgcacgaggccctgcacaacca
ctacacccagaagtccctgtccctgagccctggc.
(SEQ ID NO: 18)
TVPSSSLGTQTYICNVNHKPSNTKVDKKVEPKSCDKTHTCPPCPAPELAG
APSVFLFPPKPKDASTKGPSVFPLAPSSKSTSGGTAALGCLVKDYFPEPV
TVSWNSGALTSGVHTFPAVLQSSGLYSLSSVVTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPCREEMTKNQVSLWC
LVKGFYPSDIAVEWESNGQPENNYKTTPPVLDSDGSFFLYSKLTVDKSRW
QQGNVFSCSVMHEALHNHYTQKSLSLSPG.

In similar examples, another antibody against a second tumor associated antigen (TAA) can be the basis for generating asymmetric antibodies of the invention. The nucleotide sequence of anti-TAA-1 B1311 (an anti-Her2 antibody) heavy chain VH is as follows:

(SEQ ID NO: 5)
Gagatccagaggtgcagtaggcggaggactggctcagcctggcggctct
atcagactgagagtgcccccagcggctacatcagcagcgaccagatcct
gaactgggtcaagaaggcccctggcaagggcctggaatggatcggcaga
atctaccccgtgaccggcgtgacccagtacaaccacaagttcgtgggca
aggccaccttcagcgtggacagatccaaggacaccgtgtacatgcagat
gaacagcctgagagccgaggacaccggcgtgtactactgcggcagaggc
gagacattcgacagctggggccagggcacactgctgaccgtgtcatct.

The nucleotide sequence of anti-TAA-1 B1311 light chain VL is as follows:

(SEQ ID NO: 6)
Gacatccagctgacccagagcatcagcagcctgagcgtgtccgtgggcg
acagagtgaccatcaactgcaagagcaaccagaacctgctgtggagcgg
caacagacggtacaccctcgtgtggcaccagtggaagcctggcaagagc
cccaagcccctgatcacatgggccagcgacagatccttcggcgtgccca
gcagattcagcggcagcggctctgtgaccgacttcaccctgaccatcag
ctccgtgcagcccgaggacttcgccgtgtacttctgccagcagcacctg
gacatcccttacaccttcggcggaggcaccaagaggaaatcaagaga.

The nucleotide sequence of anti-TAA-1 B1311 ScFv is as follows:

(SEQ ID NO: 7)
gacatccagctgacccagagcatcagcagcctgagcgtgtccgtgggcga
cagagtgaccatcaactgcaagagcaaccagaacctgctgtggagcggca
acagacggtacaccdcgtgtggcaccagtggaagcctggcaagagcccca
agcccctgatcacatgggccagcgacagatccttcggcgtgcccagcaga
ttcagcggcagcggctctgtgaccgacttcaccctgaccatcagctccgt
gcagcccgaggacttcgccgtgtacttctgccagcagcacctggacatcc
cttacaccttcggcggaggcaccaagctggaaatcaagagatgtggaggc
ggttcaggcggaggtggctctggcggtggcggatcggagatccagctggt
gcagtaggcggaggactggctcagcctggcggctctatcagactgagctg
tgcccccagcggctacatcagcagcgaccagatcctgaactgggtcaaga
aggcccctggcaagggcctggaatggatcggcagaatctaccccgtgacc
ggcgtgacccagtacaaccacaagttcgtgggcaaggccaccttcagcgt
ggacagatccaaggacaccgtgtacatgcagatgaacagcctgagagccg
aggacaccggcgtgtactactgcggcagaggcgagacattcgacagctgg
ggccagggcacactgctgaccgtgtcatct.

In yet another example, Herceptin antibody may be the basis for constructing an asymmetric antibody of the invention. In this example, an ScFv based on Herceptin has the nucleotide sequence as follows:

(SEQ ID NO: 8)
Gatatccagatgacccagtccccctcctccctgtctgcctccgtgggcga
cagagtgaccatcacctgtcgggcctcccaggatgtgaacaccgccgtgg
cctggtatcagcagaagcctggcaaggcccctaagctgctgatctactcc
gcctccttcctgtactccggcgtgccctcccggttctccggctctagatc
cggcacagacttcaccctgaccatctccagcctgcagcctgaggacttcg
ccacctactactgccagcagcactacaccacccctccaaccttcggccag
ggcaccaaggtggagatcaagcggtgtggaggcggtagcggcggaggagg
atccgggggcggcgggtccggcggtggcggaagcgaggtgcagctggtgg
agtctgggggaggactggtgcagcctggcggctccctgagactgtcttgc
gctgctagcggatcaacatcaaggacacctacatccactgggttcgccag
gctccaggcaagggactggaatgggtggcccggatctaccctaccaacgg
ctacaccagatacgccgactccgtgaagggccggttcaccatctccgccg
acacctccaagaacaccgcctacctgcagatgaactccctgagggccgag
gacaccgccgtgtactactgctccagatggggaggcgacggcttctacgc
catggactactggggccagggcaccctggttaccgtgtcctcc.

Some embodiments of the invention may have a ligand (e.g., a growth factor or a cytokine) as the targeting domain. As a specific example, EGF may be used to target EGF receptor on cancer cells. The nucleotide and amino-acid sequences for EGF are as follows:

(SEQ ID NO: 9)
aatagcgatagcgagtgccctctgagccacgacggctactgtagcatgat
ggcgtgtgcatgtacatcgaggccaggataagtacgcctgcaactgcgtc
gtgggctacatcggagagagatgccagtaccgggacctgaagtggtggga
gcttaga.
(SEQ ID NO: 22)
NSDSECPLSHDGYCLHDGVCMYIEALDKYACNCVVGYIGERCQYRDLKWW
ELR.

Some embodiments of the invention may have a peptide targeting specific binder (e.g., a receptor). Any know peptide ligands may be used. Examples of peptide ligands may include AMG386 (trebananib), which is an antagonist of angiopoietin. The nucleotide and amino acid sequences for AMG386 are as follows:

(SEQ ID NO: 10)
Atgggtgcccagcaagaggaatgcgaatgggacccttggacctgcgagca
catgcttgaa.
(SEQ ID NO: 19)
MGAQQEECEWDPWTCEHMLE.

The other cancer targeting peptides (CTPs) may include the following CTP1 (targeting breast cancer) and CTP2 (targeting ovarian cancer), the nucleotide and amino acid sequences of these CTPs are as follows:

CTP1:
(SEQ ID NO: 11)
tctatggacccattcctgtttcagctgctgcagctc;
CTP1:
(SEQ ID NO: 20)
SMDPFLFQLLQL;
CTP2:
(SEQ ID NO: 12)
atgcctcatcctaccaagaacttcgacctgtacgtg;
CTP2:
(SEQ ID NO: 21)
MPHPTKNFDLYV.

An AHFS of the invention may contain an anti-CD3 ScFv fused to the C-terminus of the antibody. A linker may be used between the anti-CD3 ScFv and the CH3 domain of the antibody. An example nucleotide and amino acid sequences of a linker are as follows:

(SEQ ID NO: 13)
ggcggaggcggaggatctggtggtggtggatctggcggcggaggaagt.
(SEQ ID NO: 23)
GGGGSGGGGSGGGGS.

For T-cell targeting, an example is that of anti-CD3 ScFv. The nucleotide and amino acid sequences of OKTF1 anti-CD3 ScFv are as follows:

(SEQ ID NO: 14)
caggtgcagctggtgcagagcggcgctgaagtgaagaaacctggcgcctc
cgtgaaggtgtcctgcaaggcttctggctacacctttacccggtacacca
tgcattgggtgcgacaggctccaggccaggggctggaatggattggctac
atcaaccccagccggggctacaccaactacaatcagaagttcaaggataa
ggccaccctgaccaccgacaagtccatctccaccgcctacatggaactgt
cccggctgagatccgacgataccgctgtgtactactgcgcccggtactac
gacgaccactacaccctggactactggggacagggtactctcgtgactgt
gtcaagtggcggtggtggtagtggcgggggaggttcaggggggggaggaa
gcgaaatcgtgctgacacagagccccgccaccctgtcactgtctccaggc
gagagagctaccctgagctgactgcctcctcctccgtgtcttacatgaac
tggtatcagcagaagcccggccaggcccccagacggtggatctacgatac
ctccaagctggcctccggcatccctgccagattctccggctaggctccgg
cacctcctataccctgacaatctccagcctggaacccgaggactttgccg
tgtattactgccagcagtggtcctccaaccccttcaccttcggacagggc
acaaaggtggaaatcaagcgc.
(SEQ ID NO: 24)
QVQLVQSGAEVKKPGASVKVSCKASGYTFTRYTMHWVRQAPGQGLEWIGY
INPSRGYTNYNQKFKDKATLTTDKSISTAYMELSRLRSDDTAVYYCARYY
DDHYTLDYWGQGTLVTVSSGGGGSGGGGSGGGGSEIVLTQSPATLSLSPG
ERATLSCSASSSVSYMNWYQQKPGQAPRRWIYDTSKLASGIPARFSGSGS
GTSYTLTISSLEPEDFAVYYCQQWSSNPFTFGQGTKVEIKR.

### Example 3

### Antibody Expression and Purification

For antibody production, various clones may be expressed in any suitable cells, such as CHO or F293 cells. As an example, F293 cells (Life technologies) were transfected with the anti-TAA mAb expressing plasmid and cultured for 7 days. The anti-TAA antibody may be purified from the culture medium using a protein A affinity column (GE). Protein concentrations may be determined with a Bio-Rad protein assay kit and analyzed with 12% SDS-PAGE, using procedures known in the art or according to the manufacturer's instructions.

The various antibodies of the invention may be analyzed with techniques known in the art, such as SDS-PAGE and HPLC. For example, the solutions of anti-TAA samples may be analyzed by using a 4-12% non-reducing and reducing SDS-PAGE gel followed by Coomassie brilliant blue staining.

### Example 4

### Binding Assays

The binding affinities of antibodies of the invention may be assessed with any suitable methods known in the art, such as ELISA or Biacore. In addition, the binding may be qualitatively assessed using FACS.

Briefly, cells are harvested and washed with ice cold staining buffer (1×PBS, 1% BSA) under 1-5×10E6/ml cell density in polystyrene round bottom 12×75 mm² tubes. Cells are stained with appropriate antibodies with specific fluorescence. After staining, cells can be centrifuged to separate supernatant fluid with little loss of cells, but not so hard that the cells are difficult to suspend again.

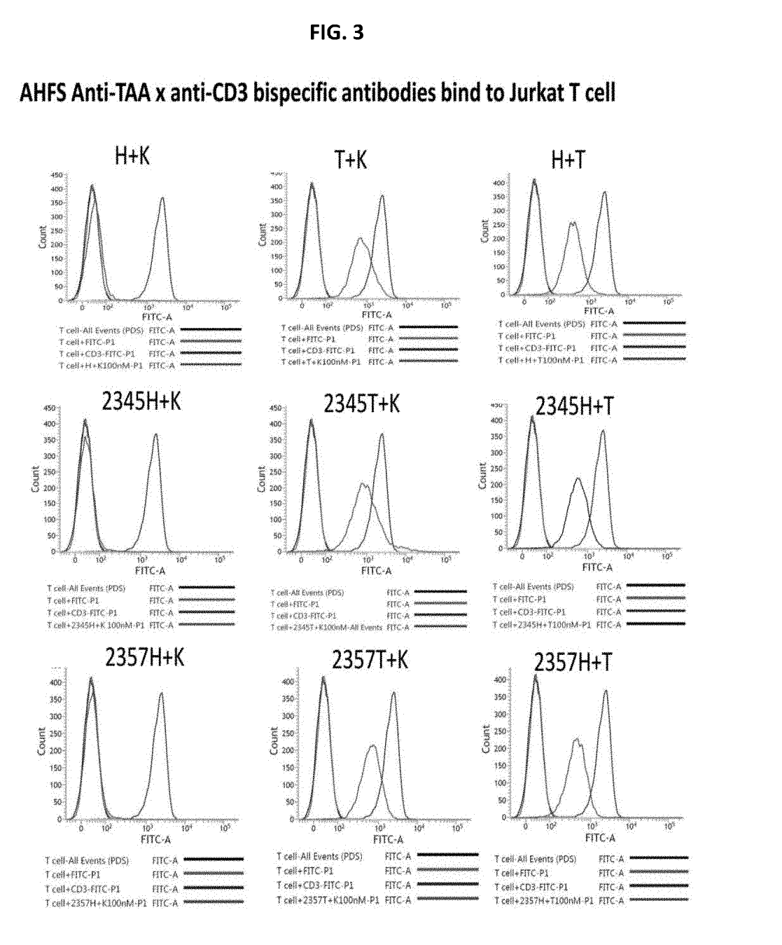

FIG. 3 shows results of FACS analysis. As shown, the knob and hole antibody (H+K) without anti-CDs ScFv did not bind Jurkat T cells. On the other hands, the asymmetric antibodies with a T-cell targeting domain (T+K, or H+T), with or without mutations in the effector binding site, bind specifically to Jurkat T cells.

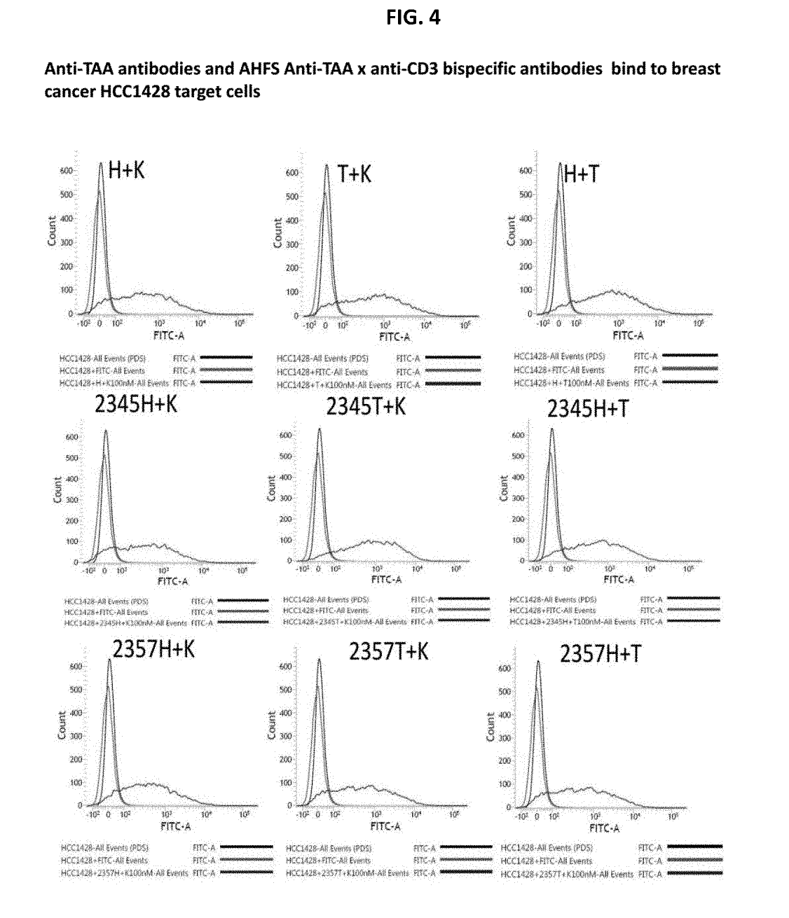

FIG. 4 shows results of FACS analysis. As shown, the knob and hole antibody (H+K) without anti-CDs ScFv did not bind breast cancer HCC1428 cells. On the other hands, the asymmetric antibodies with a T-cell targeting domain (T+K, or H+T), with or without mutations in the effector binding site, bind specifically to HCC1428 cells.

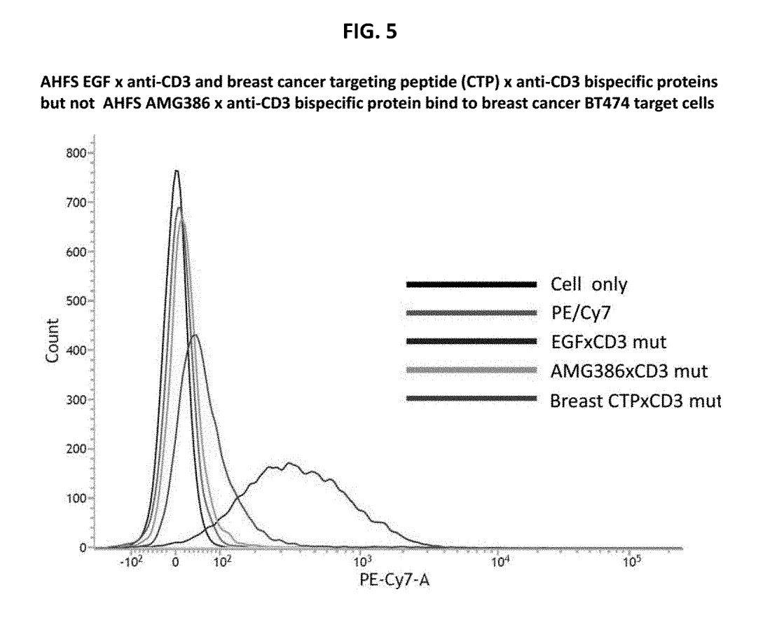

FIG. 5 shows results of FACS analysis of binding of bispecific proteins to breast cancer cells BT474. As shown, AHFS EGF×anti-CD3 and CTP targeting breast cancer cells×anti-CD3 can bind to breast cancer cells BT474, whereas AHFS AMG386×anti-CD3 cannot.

These results indicate that asymmetric antibodies (proteins) or the invention can bind specifically to the target as designed.

### Example 5

### AHFS Anti-TAA×Anti-CD3 Bispecific Antibody Effectively Kills TAA Expressing Breast Cancer Cell Line HCC1428 in the Presence of PBMC and in the Absence of ADCC Function

Abilities of the antibodies of the invention to kill cancer cells may be assessed using any suitable cells, such as MCF-7, HCC-1428, BT-474 cells, which can be obtained from ATCC. As an example, HCC1428 cells (with green fluorescence protein transfection) are cultured in a suitable culture medium at 37° C. in a humidified incubator atmosphere of 5% CO₂. All cell lines were subcultured for at least three passage, cells were plated in 96-well black flat bottom plates (10,000 cells/1000 well for all cell lines) and allowed to adhere overnight at 37° C. in a humidified atmosphere of 5% CO₂.

A solution of AHFS anti-TAA with anti-CD3 bispecific antibody is prepared and diluted into appropriated working concentrations 24 h after cell seeding. Aliquots of the AHFS anti-TAA×anti-CD3 solution were added to cell culture to achieve 20 nM and 100 nM and the cells incubated for 72 hours. PBMC or T-cells are used as effector cells at a ratio of 10:1 to the target cells. Cells are examined for green fluorescence at 0 hr and 72 hrs.

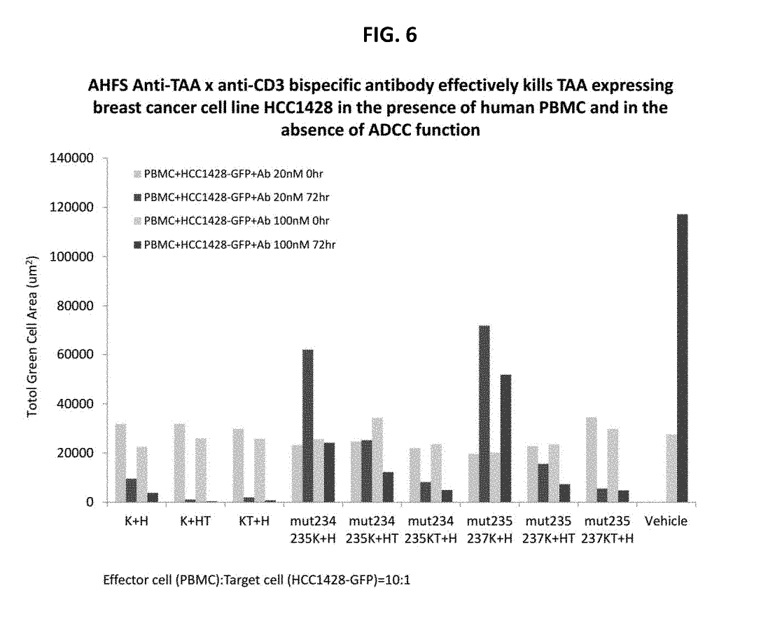

FIG. 6 shows the results of this experiment using PBMC as the effector cells. The results show that AHFS Anti-TAA×anti-CD3 bispecific antibodies effectively kills TAA-expressing breast cancer cell line HCC1428 in the presence of PBMC. The wild-type (i.e., without mutations to silence the effector functions) AHFS are able to kill cancer cells, with or without the anti-CD3 fusions. In contrast, the mutants (without the effector functions) are able to kill cancer cells and in the absence of ADCC function only with anti-CD3 fusions. That is, with mut234-235 or mut235-237, the antibodies with tethered anti-CD3 (K+HT and KT+H) are effective in killing cancer cells, while those without (K+H) are not.

Results shown in FIG. 6 clearly show that AHFS of the invention can be engineered to have minimal or no effector functions (no or little cytotoxicity with PBMC as the effector cells) and yet retain the ability to kill cancer cells via T-cell specific cytotoxicity.

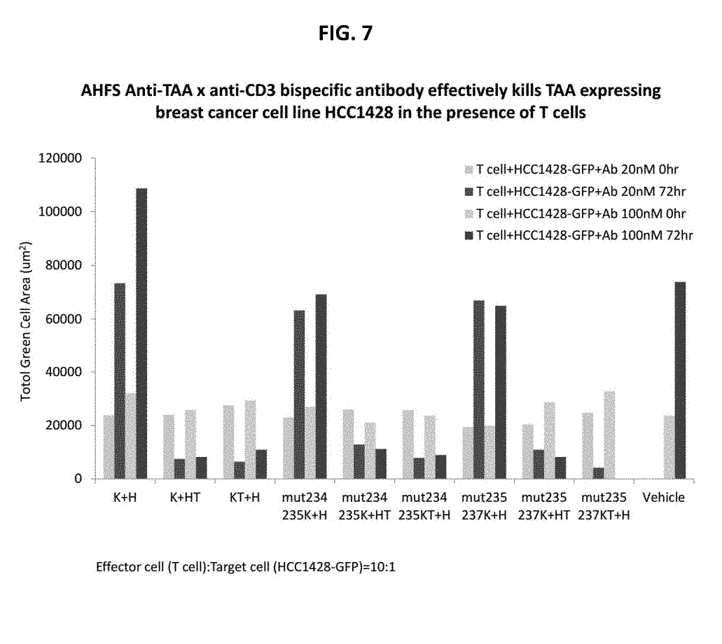

FIG. 7 shows the results of this experiment using T-cells as the effector cells. The results show that AHFS Anti-TAA×anti-CD3 bispecific antibodies effectively kills TAA-expressing breast cancer cell line HCC1428 in the presence of T-cells. The wild-type (i.e., without mutations to silence the effector functions) or mutant (mut234-235 or mut235-237) AHFS without anti-CD3 fusions are not effective in killing cancer cells. Without the anti-CD3 fusions, these antibodies cannot engage and activate T-cells.

Results shown in FIG. 7 clearly show that AHFS of the invention can be engineered (anti-CD3 fusion) to depend on T-cell engagement and activation, thereby avoiding non-specific ADCC.

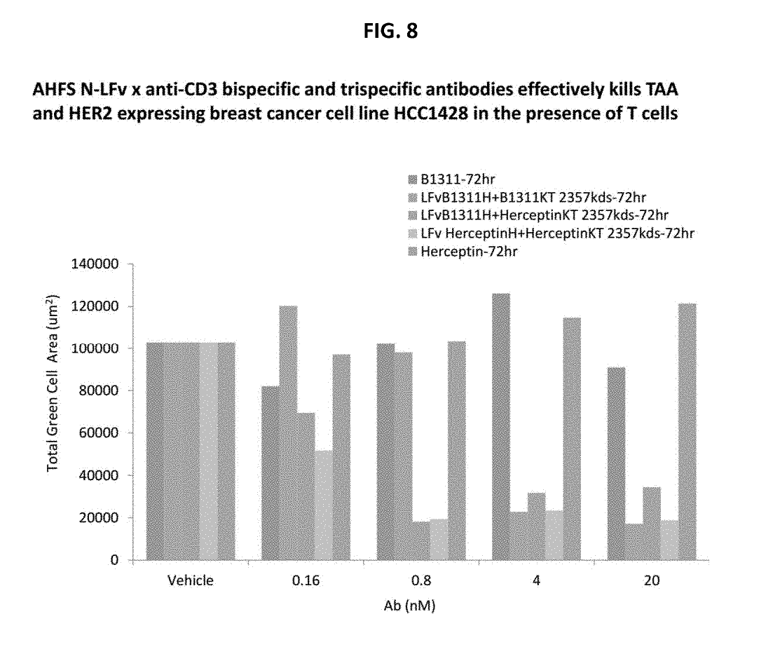

FIG. 8 shows the results of a similar experiment using T-cells as the effector cells and a new format (N-LFv) of an asymmetric antibody. The results show that AHFS N-LFv×anti-CD3 bispecific antibodies effectively kills Her2 expressing breast cancer cell line HCC1428 in the presence of T-cells. In contrast, both B1311 and Herceptin are not effective due to the lack of ADCC (no NK cells in this assay).

Results shown in FIG. 8 clearly show that AHFS of the invention can be engineered (anti-CD3 fusion) to depend on T-cell engagement and activation and not depend on the effector functions, thereby avoiding non-specific ADCC.

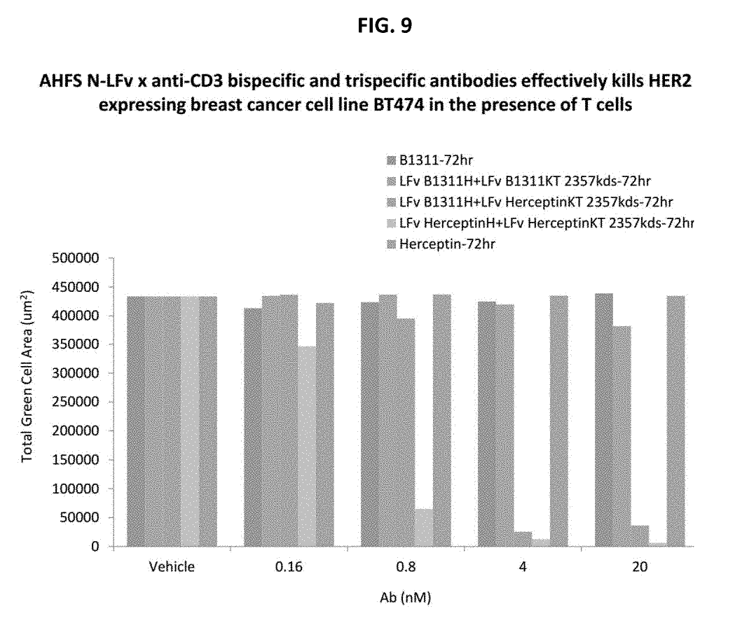

FIG. 9 shows the results of a similar experiment using T-cells as the effector cells and the same format (N-LFv) of an asymmetric antibody, but on a different cancer cell line (BT474). The results show that AHFS N-LFv×anti-CD3 bispecific antibodies effectively kills Her2 expressing breast cancer cell line BT474 in the presence of T-cells. In contrast, B1311 and Herceptin are not effective due to the absence of effector functions (no NK cells).

Results shown in FIG. 9 clearly show that AHFS of the invention can be engineered (anti-CD3 fusion) to depend on T-cell engagement and activation and not depend on the effector functions, thereby avoiding non-specific ADCC.

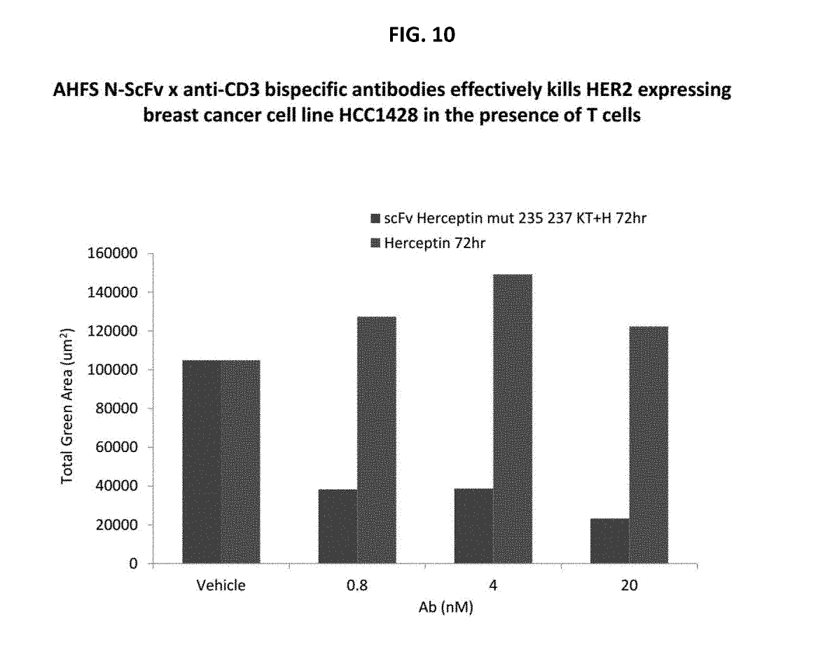

FIG. 10 shows the results of a similar experiment using T-cells as the effector cells and a new format (N-ScFv) of an asymmetric antibody. The results show that AHFS N-ScFv×anti-CD3 bispecific antibodies effectively kills Her2 expressing breast cancer cell line HCC1428 in the presence of T-cells, without NK cells. In contrast, Herceptin is not effective due to the absence of NK cells.

Results shown in FIG. 10 clearly show that AHFS of the invention can be engineered (anti-CD3 fusion) to depend on T-cell engagement and activation, thereby avoiding non-specific ADCC.

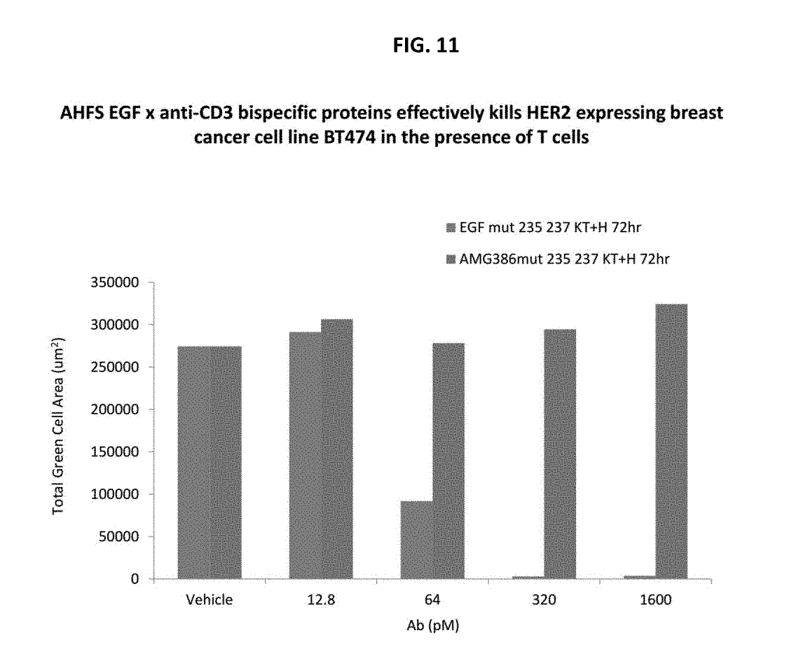

In addition to binding domains based on antibodies, embodiments of the invention can also be based on ligands (e.g., growth factors or cytokines) to target the cancer cells. FIG. 11 shows the results of an experiment using T-cells as the effector cells and a new format (EGF) of an asymmetric antibody. The results show that AHFS EGF×anti-CD3 bispecific antibodies effectively kills Her2 expressing breast cancer cell line BT474 in the presence of T-cells, without NK cells. In contrast, AMG386-based bispecific antibody is not effective due to the absence of NK cells. AMG386 binds to angiopoietin, which is not present on BT474.

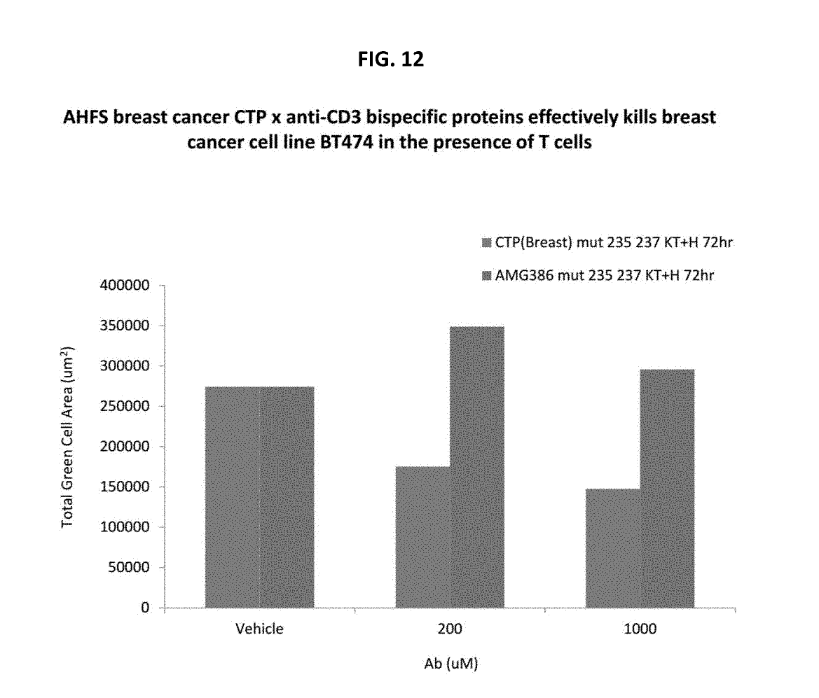

Some embodiments of the invention are based on peptide ligands that can target tumor cells. FIG. 12 shows the results of an experiment using T-cells as the effector cells and an asymmetric antibody having a peptide that targets cancer cells (CTP). The results show that AHFS CTP×anti-CD3 bispecific antibodies effectively kills breast cancer cell line BT474 in the presence of T-cells, without NK cells. In contrast, AMG386-based bispecific antibody is not effective due to the absence of NK cells. AMG386 binds to angiopoietin, which is not present on BT474.

The results from the above experiments clearly demonstrate the utility of bispecific or trispecific antibodies. Antibodies of the invention can have targeting domains (Binder A and Binder B in FIG. 1A) based on other antibodies. These binder domains can be in the form of regular variable domains, Fab, LFv, or ScFv. In addition, these binder domains can be based on a ligand (e.g., growth factors or cytokines) or a cancer-targeting peptide. Antibodies of the invention have a specific T-cell targeting domain (e.g., anti-CD3 ScFv) that can engage and activate T-cells. In addition, asymmetric antibodies (or asymmetric proteins) of the invention may have mutations in the effector binding sites such that the effector functions are diminished or abolished, thereby minimizing non-specific T cell actions.

### Example 6

### Non-Specific T Cell Activation is Prevented by Silencing the Effector Functions

The AHFS multi-specific antibodies of the invention are engineered to have little or no effector functions such that non-specific T-cell engagement and activation can be avoided. T-cell activation produces cytokines (e.g., IL-2, TNF-α, INF-γ) and other factors (e.g., perforin, granzyme A, granzyme B, etc.). Without the effector functions, AHFS multi-specific antibodies of the invention will not induce non-specific T-cell activation. That is, T-cells activation with these antibodies depend on specific binding of the antibodies to the target cancer cells.

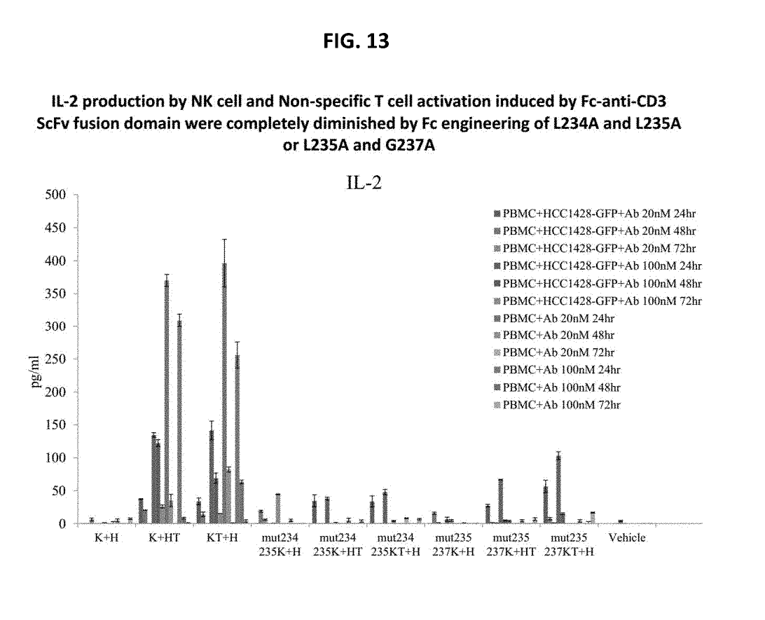

FIG. 13 shows that IL-2 production by T-cells is diminished when treated with AHFS multi-specific antibodies of the invention without the effector functions (i.e., mutants of L234A and L235A or L235A and G237A). In contrast, AHFS multi-specific antibodies of the invention with native effector functions (K+HT or KT+H) can still induce IL-2 production in the presence of PBMC. As noted above, with diminished effector functions, AHFS multi-specific antibodies of the invention can avoid non-specific T cells actions.

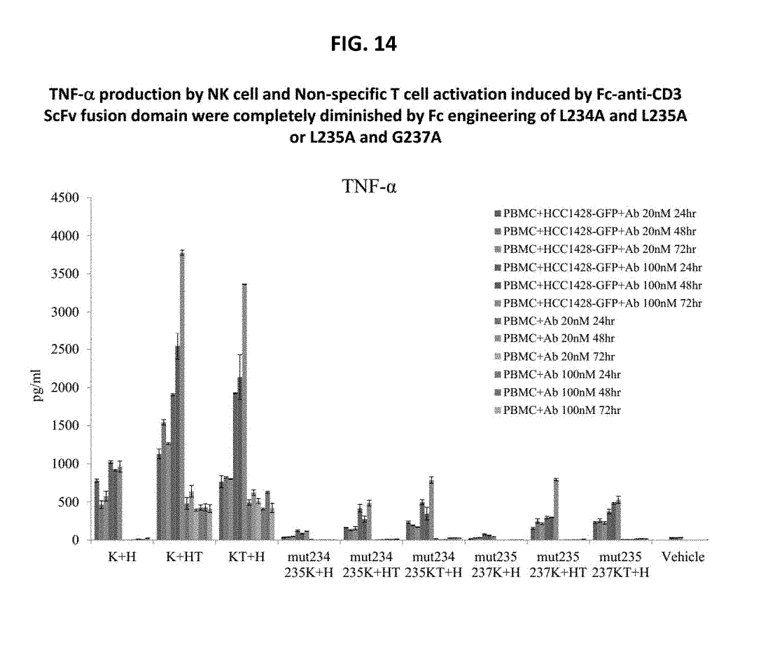

FIG. 14 shows that TNF-α production by T-cells is diminished when treated with AHFS multi-specific antibodies of the invention without the effector functions (i.e., mutants of L234A and L235A or L235A and G237A).

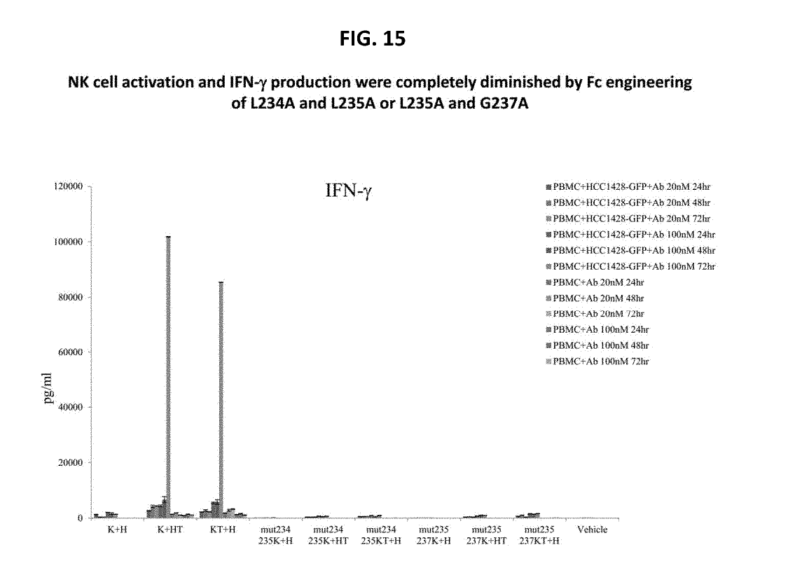

FIG. 15 shows that INF-γ production by T-cells is completely abolished when treated with AHFS multi-specific antibodies of the invention without the effector functions (i.e., mutants of L234A and L235A or L235A and G237A).

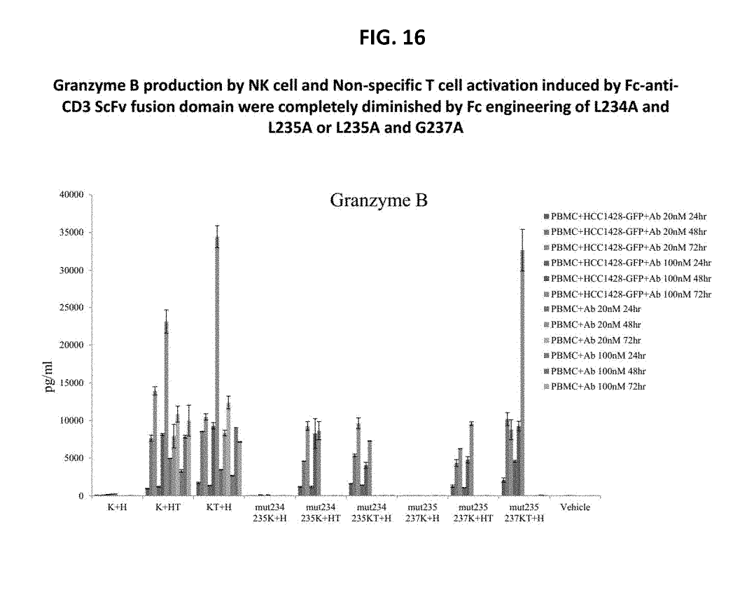

FIG. 16 shows that Granzyme B production by T-cells and non-specific T cell activation are diminished when treated with AHFS multi-specific antibodies of the invention without the effector functions (i.e., mutants of L234A and L235A or L235A and G237A). Granzyme B is secreted by NK cells along with the perforin to mediate apoptosis in the target cells.

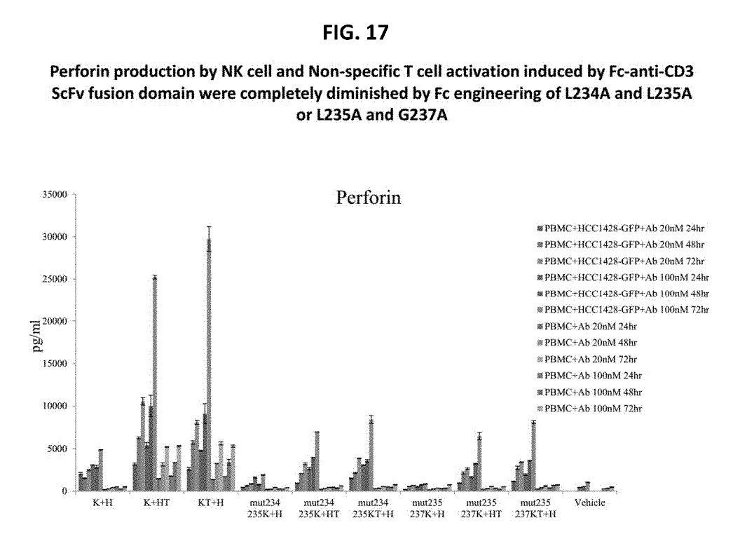

FIG. 17 shows that perforin production by T-cells and non-specific T cell activation are diminished when treated with AHFS multi-specific antibodies of the invention without the effector functions (i.e., mutants of L234A and L235A or L235A and G237A).

Results shown in FIGS. 13-17 clearly indicate that non-specific T-cell activation and NK cell actions can be avoided with AHFS multi-specific antibodies (with mutations in the effector binding site) of the invention. Therefore, T-cells mediated action will be dependent on specific binding of the AHFS multi-specific antibodies the target cells, thereby achieving the therapeutic effects without the undesired effects.

### Example 7

### Specific T Cell Activation Depends on Target Cell Binding

The AHFS multi-specific antibodies of the invention are engineered to have little or no effector functions such that non-specific T-cell engagement and activation can be avoided. As a result, T-cell activation by AHFS multi-specific antibodies of the invention depends on specific binding of the antibodies to the target cancer cells.

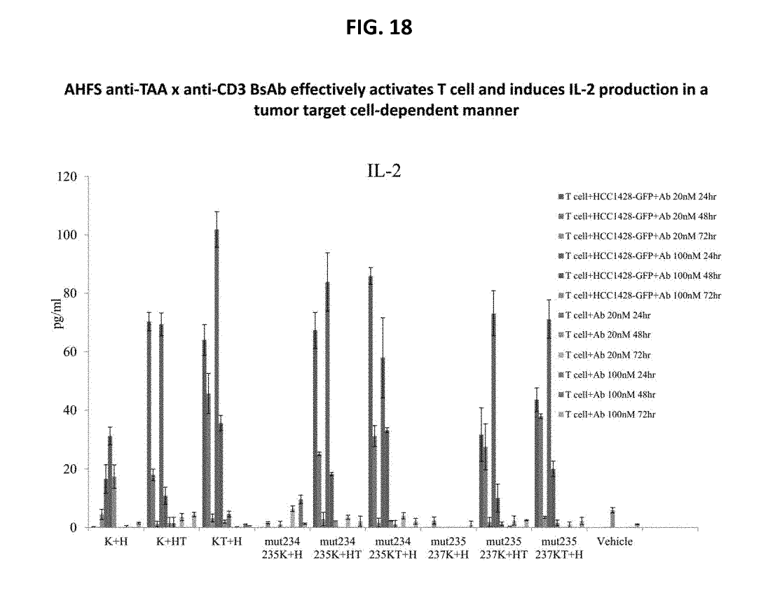

FIG. 18 shows that in the presence of the target tumor cells (HCC1428), AHFS multi-specific antibodies of the invention without the effector functions (i.e., mutants of L234A and L235A or L235A and G237A) can induce the production of IL-2 by T-cells. In contrast, in the absence of the target tumor cells, AHFS multispecific antibodies of the invention do not induce the production of IL-2. This result indicates that engagement of the target tumor cells is necessary for T cell activation using the AHFS multispecific antibodies of the invention.

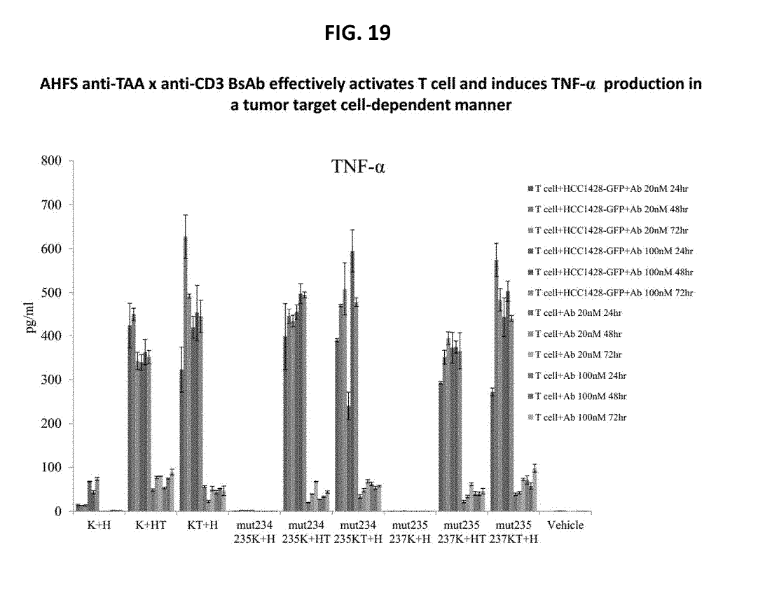

FIG. 19 shows that in the presence of the target tumor cells, AHFS multi-specific antibodies of the invention without the effector functions (i.e., mutants of L234A and L235A or L235A and G237A) can induce the production of TNF-α by T-cells. In contrast, in the absence of the target tumor cells, AHFS multispecific antibodies of the invention do not induce the production of TNF-α. This result indicates that engagement of the target tumor cells is necessary for T cell activation using the AHFS multispecific antibodies of the invention.

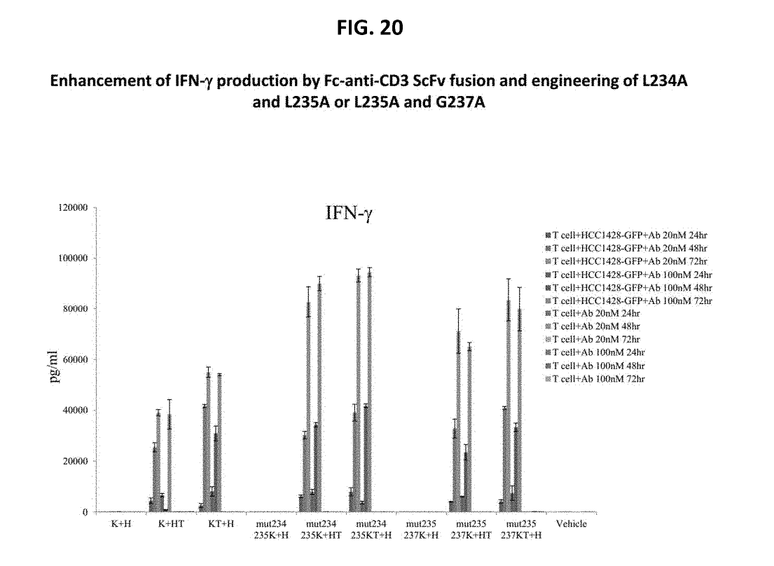

FIG. 20 shows that in the presence of the target tumor cells, AHFS multi-specific antibodies of the invention without the effector functions (i.e., mutants of L234A and L235A or L235A and G237A) can induce the production of INF-γ by T-cells. In contrast, in the absence of the target tumor cells, AHFS multispecific antibodies of the invention do not induce the production of INF-γ. This result indicates that engagement of the target tumor cells is necessary for T cell activation using the AHFS multispecific antibodies of the invention.

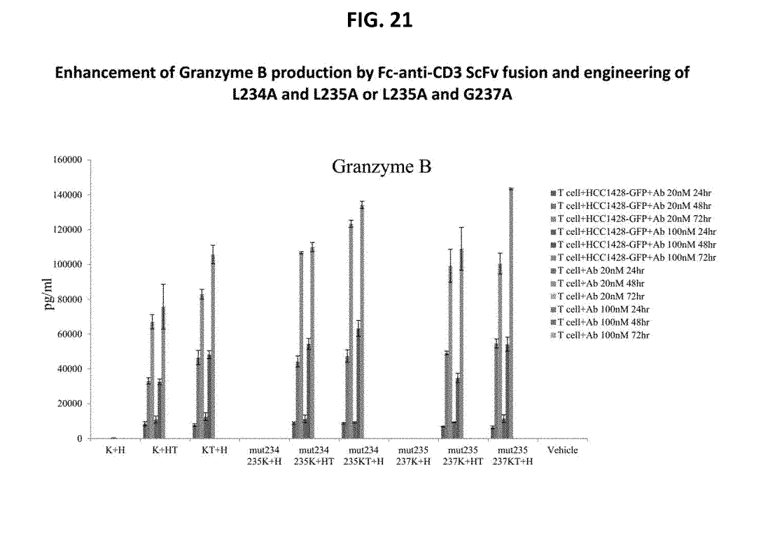

FIG. 21 shows that in the presence of the target tumor cells, AHFS multi-specific antibodies of the invention without the effector functions (i.e., mutants of L234A and L235A or L235A and G237A) can induce the production of Granzyme B by T-cells. In contrast, in the absence of the target tumor cells, AHFS multispecific antibodies of the invention do not induce the production of Granzyme B. This result indicates that engagement of the target tumor cells is necessary for T cell activation using the AHFS multispecific antibodies of the invention.

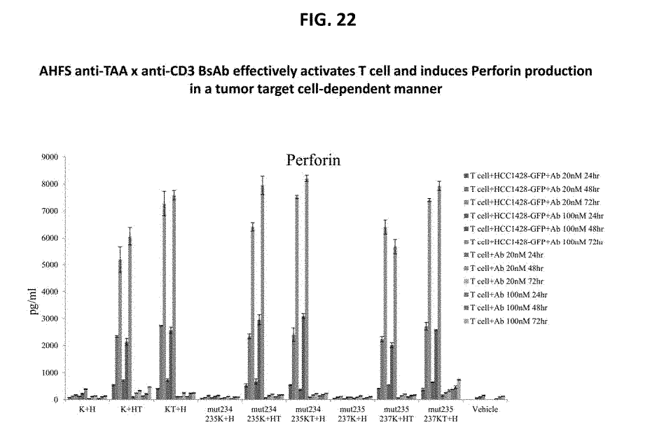

FIG. 22 shows that in the presence of the target tumor cells, AHFS multi-specific antibodies of the invention without the effector functions (i.e., mutants of L234A and L235A or L235A and G237A) can induce the production of perforin by T-cells. In contrast, in the absence of the target tumor cells, AHFS multispecific antibodies of the invention do not induce the production of perforin. This result indicates that engagement of the target tumor cells is necessary for T cell activation using the AHFS multispecific antibodies of the invention.

Results shown in FIGS. 18-22 clearly indicate that while non-specific T-cell activation can be avoided with AHFS multi-specific antibodies of the invention, these antibodies can engage and activate T-cells to have specific T-cell cytotoxicity in a target cell dependent manner. These results indicate that as therapeutics, AHFS multispecific antibodies of the invention can be more specific and have less undesirable effects.

Some embodiments of the invention relate to methods of treating cancers using any one of the AHFS multispecific antibodies of the invention. The cancers that can be treated with embodiments of the invention are not particular limited as long as one can design a specific binding domain or ligand to target a tumor-associated antigen, as evidenced by the various cancers cells shown above.

While embodiments of the invention have been illustrated with limited number of examples, one skilled in the art would appreciate that other modifications and variations are possible without departing from the scope of the invention. Therefore, the scope of protection of this invention should only be limited by the attached claims.

1. An asymmetric heterodimeric antibody, comprising:
a knob structure formed in a CH3 domain of a first heavy chain;
a hole structure formed in a CH3 domain of a second heavy chain, wherein the hole structure is configured to accommodate the knob structure so that a heterodimeric antibody is formed; and
a T-cell targeting domain fused to the CH3 domain of the first heavy chain or the second heavy chain, wherein the T-cell targeting domain binds specifically to an antigen on the T-cell.

2. The asymmetric heterodimeric antibody according to claim 1, wherein the T-cell targeting domain is a ScFv or Fab.

3. The asymmetric heterodimeric antibody according to claim 1, wherein the ScFv or Fab is derived from an anti-CD3 antibody.

4. The asymmetric heterodimeric antibody according to claim 1, wherein a first binding or targeting domain of the asymmetric heterodimeric antibody binds specifically to a tumor associated antigen.

5. The asymmetric heterodimeric antibody according to claim 4, wherein the asymmetric heterodimeric antibody comprises a second binding or targeting domain that is different from the first binding or targeting domain.

6. The asymmetric heterodimeric antibody according to claim 4, wherein the asymmetric heterodimeric antibody comprises a second binding or targeting domain that is identical to the first binding or targeting domain.

7. The asymmetric heterodimeric antibody according to claim 1, further comprising mutations at an effector binding site such that the asymmetric heterodimeric antibody has a diminished effector function.

8. The asymmetric heterodimeric antibody according to claim 7, wherein the mutations comprise L234A and L235A mutations.

9. The asymmetric heterodimeric antibody according to claim 7, wherein the mutations comprise L235A and G237A mutations.

10. A pharmaceutical composition for treating cancer comprising the asymmetric heterodimeric antibody according to claim 1.

11. A method for treating cancer comprising: administering to a subject in need thereof the pharmaceutical composition of claim 10.

12. The method of claim 11, wherein the cancer is breast cancer or ovarian cancer.


SEQUENCE LISTING (24 sequences)

SEQ ID NO: 1 - DNA, 987 bp, Artificial Sequence
  Synthetic
gctagcaccaagggcccttccgtgttccctctggccccttccagcaagtctacctctggcggcaccgctgctctgggctgcctcgtgaaggactacttccctgagcctgtgacagtgtcctggaactctggcgccctgacctccggcgtgcacaccttccctgccgtgctgcagtcctccggcctgtactctctgtcctccgtcgtgacagtgccttcctccagcctgggcacccagacctacatctgcaacgtgaaccacaagccttccaacaccaaggtggacaagaaggtggagcctaagtcctgcgacaagacccacacctgtcctccatgccctgcccctgaggctgctggcggaccctccgtgttcctgttccctccaaagcctaaggacaccctgatgatctcccggacccctgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaacctcaagtgtgcaccctgccccctagccgggaagagatgaccaagaaccaggtgtccctgtcctgcgccgtgaagggcttctacccctccgacattgccgtggaatgggagtccaacggccagcctgagaacaactacaagaccaccccccctgtgctggactccgacggctcattcttcctggtgtccaagctgacagtggacaagtcccggtggcagcagggcaacgtgttctcctgctccgtgatgcacgaggccctgcacaaccactacacccagaagtccctgagcctgtcccctggc

SEQ ID NO: 2 - DNA, 987 bp, Artificial Sequence
  Synthetic
gctagcaccaagggcccttccgtgttccctctggccccttccagcaagtctacctctggcggcaccgctgctctgggctgcctcgtgaaggactacttccctgagcctgtgacagtgtcctggaactctggcgccctgacctccggcgtgcacaccttccctgccgtgctgcagtcctccggcctgtactctctgtcctccgtcgtgacagtgccttcctccagcctgggcacccagacctacatctgcaacgtgaaccacaagccttccaacaccaaggtggacaagaaggtggagcctaagtcctgcgacaagacccacacctgtcctccatgccctgcccctgaggctgctggcggaccctccgtgttcctgttccctccaaagcctaaggacaccctgatgatctcccggacccctgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgcccccttgccgggaagagatgaccaagaaccaggtgtccctgtggtgcctcgtgaagggcttctacccctccgacattgccgtggaatgggagtccaacggccagcctgagaacaactacaagaccaccccccctgtgctggactccgacggctcattcttcctgtactccaagctgacagtggacaagtcccggtggcagcagggcaacgtgttctcctgctccgtgatgcacgaggccctgcacaaccactacacccagaagtccctgtccctgagccctggc

SEQ ID NO: 3 - DNA, 987 bp, Artificial Sequence
  Synthetic
gctagcaccaagggcccttccgtgttccctctggccccttccagcaagtctacctctggcggcaccgctgctctgggctgcctcgtgaaggactacttccctgagcctgtgacagtgtcctggaactctggcgccctgacctccggcgtgcacaccttccctgccgtgctgcagtcctccggcctgtactctctgtcctccgtcgtgacagtgccttcctccagcctgggcacccagacctacatctgcaacgtgaaccacaagccttccaacaccaaggtggacaagaaggtggagcctaagtcctgcgacaagacccacacctgtcctccatgccctgcccctgagctggctggcgctccctccgtgttcctgttccctccaaagcctaaggacaccctgatgatctcccggacccctgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaacctcaagtgtgcaccctgccccctagccgggaagagatgaccaagaaccaggtgtccctgtcctgcgccgtgaagggcttctacccctccgacattgccgtggaatgggagtccaacggccagcctgagaacaactacaagaccaccccccctgtgctggactccgacggctcattcttcctggtgtccaagctgacagtggacaagtcccggtggcagcagggcaacgtgttctcctgctccgtgatgcacgaggccctgcacaaccactacacccagaagtccctgagcctgtcccctggc

SEQ ID NO: 4 - DNA, 987 bp, Artificial Sequence
  Synthetic
gctagcaccaagggcccttccgtgttccctctggccccttccagcaagtctacctctggcggcaccgctgctctgggctgcctcgtgaaggactacttccctgagcctgtgacagtgtcctggaactctggcgccctgacctccggcgtgcacaccttccctgccgtgctgcagtcctccggcctgtactctctgtcctccgtcgtgacagtgccttcctccagcctgggcacccagacctacatctgcaacgtgaaccacaagccttccaacaccaaggtggacaagaaggtggagcctaagtcctgcgacaagacccacacctgtcctccatgccctgcccctgagctggctggcgctccctccgtgttcctgttccctccaaagcctaaggacaccctgatgatctcccggacccctgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgcccccttgccgggaagagatgaccaagaaccaggtgtccctgtggtgcctcgtgaagggcttctacccctccgacattgccgtggaatgggagtccaacggccagcctgagaacaactacaagaccaccccccctgtgctggactccgacggctcattcttcctgtactccaagctgacagtggacaagtcccggtggcagcagggcaacgtgttctcctgctccgtgatgcacgaggccctgcacaaccactacacccagaagtccctgtccctgagccctggc

SEQ ID NO: 5 - DNA, 345 bp, Mus musculus
gagatccagctggtgcagtctggcggaggactggctcagcctggcggctctatcagactgagctgtgcccccagcggctacatcagcagcgaccagatcctgaactgggtcaagaaggcccctggcaagggcctggaatggatcggcagaatctaccccgtgaccggcgtgacccagtacaaccacaagttcgtgggcaaggccaccttcagcgtggacagatccaaggacaccgtgtacatgcagatgaacagcctgagagccgaggacaccggcgtgtactactgcggcagaggcgagacattcgacagctggggccagggcacactgctgaccgtgtcatct

SEQ ID NO: 6 - DNA, 342 bp, Mus musculus
gacatccagctgacccagagcatcagcagcctgagcgtgtccgtgggcgacagagtgaccatcaactgcaagagcaaccagaacctgctgtggagcggcaacagacggtacaccctcgtgtggcaccagtggaagcctggcaagagccccaagcccctgatcacatgggccagcgacagatccttcggcgtgcccagcagattcagcggcagcggctctgtgaccgacttcaccctgaccatcagctccgtgcagcccgaggacttcgccgtgtacttctgccagcagcacctggacatcccttacaccttcggcggaggcaccaagctggaaatcaagaga

SEQ ID NO: 7 - DNA, 732 bp, Artificial Sequence
  Synthetic
gacatccagctgacccagagcatcagcagcctgagcgtgtccgtgggcgacagagtgaccatcaactgcaagagcaaccagaacctgctgtggagcggcaacagacggtacaccctcgtgtggcaccagtggaagcctggcaagagccccaagcccctgatcacatgggccagcgacagatccttcggcgtgcccagcagattcagcggcagcggctctgtgaccgacttcaccctgaccatcagctccgtgcagcccgaggacttcgccgtgtacttctgccagcagcacctggacatcccttacaccttcggcggaggcaccaagctggaaatcaagagatgtggaggcggttcaggcggaggtggctctggcggtggcggatcggagatccagctggtgcagtctggcggaggactggctcagcctggcggctctatcagactgagctgtgcccccagcggctacatcagcagcgaccagatcctgaactgggtcaagaaggcccctggcaagggcctggaatggatcggcagaatctaccccgtgaccggcgtgacccagtacaaccacaagttcgtgggcaaggccaccttcagcgtggacagatccaaggacaccgtgtacatgcagatgaacagcctgagagccgaggacaccggcgtgtactactgcggcagaggcgagacattcgacagctggggccagggcacactgctgaccgtgtcatct

SEQ ID NO: 8 - DNA, 744 bp, Artificial Sequence
  Synthetic
gatatccagatgacccagtccccctcctccctgtctgcctccgtgggcgacagagtgaccatcacctgtcgggcctcccaggatgtgaacaccgccgtggcctggtatcagcagaagcctggcaaggcccctaagctgctgatctactccgcctccttcctgtactccggcgtgccctcccggttctccggctctagatccggcacagacttcaccctgaccatctccagcctgcagcctgaggacttcgccacctactactgccagcagcactacaccacccctccaaccttcggccagggcaccaaggtggagatcaagcggtgtggaggcggtagcggcggaggaggatccgggggcggcgggtccggcggtggcggaagcgaggtgcagctggtggagtctgggggaggactggtgcagcctggcggctccctgagactgtcttgcgctgctagcggcttcaacatcaaggacacctacatccactgggttcgccaggctccaggcaagggactggaatgggtggcccggatctaccctaccaacggctacaccagatacgccgactccgtgaagggccggttcaccatctccgccgacacctccaagaacaccgcctacctgcagatgaactccctgagggccgaggacaccgccgtgtactactgctccagatggggaggcgacggcttctacgccatggactactggggccagggcaccctggttaccgtgtcctcc

SEQ ID NO: 9 - DNA, 159 bp, Homo sapiens
aatagcgatagcgagtgccctctgagccacgacggctactgtctgcatgatggcgtgtgcatgtacatcgaggccctggataagtacgcctgcaactgcgtcgtgggctacatcggagagagatgccagtaccgggacctgaagtggtgggagcttaga

SEQ ID NO: 10 - DNA, 60 bp, Artificial Sequence
  Synthetic
atgggtgcccagcaagaggaatgcgaatgggacccttggacctgcgagcacatgcttgaa

SEQ ID NO: 11 - DNA, 36 bp, Artificial Sequence
  Synthetic
tctatggacccattcctgtttcagctgctgcagctc

SEQ ID NO: 12 - DNA, 36 bp, Artificial Sequence
  Synthetic
atgcctcatcctaccaagaacttcgacctgtacgtg

SEQ ID NO: 13 - DNA, 48 bp, Artificial Sequence
  Synthetic
ggcggaggcggaggatctggtggtggtggatctggcggcggaggaagt

SEQ ID NO: 14 - DNA, 723 bp, Artificial Sequence
  Synthetic
caggtgcagctggtgcagagcggcgctgaagtgaagaaacctggcgcctccgtgaaggtgtcctgcaaggcttctggctacacctttacccggtacaccatgcattgggtgcgacaggctccaggccaggggctggaatggattggctacatcaaccccagccggggctacaccaactacaatcagaagttcaaggataaggccaccctgaccaccgacaagtccatctccaccgcctacatggaactgtcccggctgagatccgacgataccgctgtgtactactgcgcccggtactacgacgaccactacaccctggactactggggacagggtactctcgtgactgtgtcaagtggcggtggtggtagtggcgggggaggttcaggggggggaggaagcgaaatcgtgctgacacagagccccgccaccctgtcactgtctccaggcgagagagctaccctgagctgctctgcctcctcctccgtgtcttacatgaactggtatcagcagaagcccggccaggcccccagacggtggatctacgatacctccaagctggcctccggcatccctgccagattctccggctctggctccggcacctcctataccctgacaatctccagcctggaacccgaggactttgccgtgtattactgccagcagtggtcctccaaccccttcaccttcggacagggcacaaaggtggaaatcaagcgc

SEQ ID NO: 15 - protein, 329 aa, Artificial Sequeence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 16 - protein, 329 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 329 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 18 - protein, 329 aa, Artificial Sequence
  Synthetic
TVPSSSLGTQTYICNVNHKPSNTKVDKKVEPKSCDKTHTCPPCPAPELAGAPSVFLFPPKPKDASTKGPSVFPLAPSSKSTSGGTAALGCLVKDYFPEPVTVSWNSGALTSGVHTFPAVLQSSGLYSLSSVVTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 20 aa, Artificial Sequence
  Synthetic
MGAQQEECEWDPWTCEHMLE

SEQ ID NO: 20 - protein, 12 aa, Artificial Sequence
  Synthetic
SMDPFLFQLLQL

SEQ ID NO: 21 - protein, 12 aa, Artificial Sequence
  Synthetic
MPHPTKNFDLYV

SEQ ID NO: 22 - protein, 53 aa, Homo sapiens
NSDSECPLSHDGYCLHDGVCMYIEALDKYACNCVVGYIGERCQYRDLKWWELR

SEQ ID NO: 23 - protein, 15 aa, Artificial Sequence
  Synthetic
GGGGSGGGGSGGGGS

SEQ ID NO: 24 - protein, 241 aa, Mus musculus
QVQLVQSGAEVKKPGASVKVSCKASGYTFTRYTMHWVRQAPGQGLEWIGYINPSRGYTNYNQKFKDKATLTTDKSISTAYMELSRLRSDDTAVYYCARYYDDHYTLDYWGQGTLVTVSSGGGGSGGGGSGGGGSEIVLTQSPATLSLSPGERATLSCSASSSVSYMNWYQQKPGQAPRRWIYDTSKLASGIPARFSGSGSGTSYTLTISSLEPEDFAVYYCQQWSSNPFTFGQGTKVEIKR